(19) United States
(12) Patent Application Publication
(10) Pub. No.: US 2025/0027941 A1
(43) Pub. Date: Jan. 23, 2025

(54) METHODS OF ASSAYING MICRO-OBJECTS IN A MICROFLUIDIC DEVICE

(71) Applicant: BRUKER CELLULAR ANALYSIS, INC., Emeryville, CA (US)

(72) Inventors: Ke-Chih LIN, Richmond, CA (US); Long Van LE, San Francisco, CA (US); Jason M. MCEWEN, El Cerrito, CA (US); J. Tanner NEVILL, El Cerrito, CA (US); Volker L.S. KURZ, Oakland, CA (US); Peyton SHIEH, Emeryville, CA (US); Alexander J. MASTROIANNI, Alameda, CA (US); Or GADISH, Richmond, CA (US); Ethan Jun Wei GOH, Salt Lake City, UT (US)

(73) Assignee: BRUKER CELLULAR ANALYSIS, INC., Emeryville, CA (US)

(21) Appl. No.: 18/783,636

(22) Filed: Jul. 25, 2024

Related U.S. Application Data
(63) Continuation of application No. PCT/US2023/061224, filed on Jan. 25, 2023.
(60) Provisional application No. 63/440,637, filed on Jan. 23, 2023.
(60) Provisional application No. 63/302,888, filed on Jan. 25, 2022.

(51) Int. Cl. G01N 33/569; B01L 3/00

(52) U.S. Cl. CPC G01N 33/56972; B01L 3/502715; B01L 2200/16; B01L 2300/0877; B01L 2300/0883; B01L 2300/18; G01N 2333/7051; G01N 2333/70517; G01N 2333/70596

(57) ABSTRACT

This application describes systems and methods for assaying micro-objects in a microfluidic device. These methods include contacting a reagent with a micro-object by introducing a reagent in a first fluidic medium to a flow region of a microfluidic device, wherein the microfluidic device comprises the flow region and a chamber comprising a proximal opening fluidically connecting the chamber to the flow region, diffusing the reagent from the flow region into the chamber; introducing a micro-object into the flow region of the microfluidic device, and diffusing the reagent from the chamber to the flow region to contact the reagent with the micro-object within the flow region. Other embodiments are described.

### CROSS-REFERENCE TO RELATED APPLICATIONS

The application is a continuation of International Patent Application No. PCT/US2023/061224, METHODS OF ASSAYING MICRO-OBJECTS IN A MICROFLUIDIC DEVICE, filed Jan. 25, 2023, which claims the benefit of priority to U.S. Provisional Application No. 63/302,888, METHODS OF ASSAYING MICRO-OBJECTS IN A MICROFLUIDIC DEVICE, filed on Jan. 25, 2022; and U.S. Provisional Application No. 63/440,637, COMPOSITIONS FOR MICROFLUIDIC WORKFLOWS AND METHODS OF USE THEREOF, filed on Jan. 23, 2023, each of which disclosures are herein incorporated by reference in their entirety.

### BACKGROUND

Microfluidic devices allow researchers to assay micro-objects such as biological cells using significantly less assay reagents compared with traditional laboratory assays. In addition, a microfluidic device usually is operated with a certain level of automation, which can be beneficial to reduce the time and labor required for the experiments. Nevertheless, there is still a continuous need for a more efficient way to characterize micro-objects. The present disclosure relates to methods for assaying micro-objects in a microfluidic device.

### SUMMARY

In a first aspect, a method of contacting a micro-object with a reagent within a microfluidic device is provided. The microfluidic device can comprise a microfluidic circuit material defining a flow region and a chamber comprising a proximal opening fluidically connecting the chamber to the flow region. The method can comprise introducing a micro-object into the flow region of the microfluidic device; and allowing a reagent to diffuse from the chamber to the flow region and to contact the micro-object.

In a second aspect, a method of sampling a micro-object population in a microfluidic device is provided. The microfluidic device can comprise a microfluidic circuit material defining a flow region and a chamber comprising a proximal opening fluidically connecting the chamber to the flow region. The method can comprise introducing a first fluidic medium comprising a first reagent to the flow region of the microfluidic device; and allowing the first reagent to diffuse from the flow region to the chamber; introducing a plurality of micro-objects into the flow region of the microfluidic device; allowing the first reagent to diffuse from the chamber to the flow region to contact the plurality of micro-objects; and observing an interaction between the first assay reagent and the plurality of micro-objects.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A to 2B illustrate a microfluidic device having sequestration pens according to some embodiments of the disclosure.

FIG. 2C illustrates a sequestration pen of a microfluidic device according to some embodiments of the disclosure.

FIG. 3 illustrates a sequestration pen of a microfluidic device according to some embodiments of the disclosure.

FIGS. 4A to 4B illustrate electrokinetic features of a microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIG. 6 illustrates an embodiment of assaying micro-objects in a microfluidic device according to the present disclosure.

FIG. 7 illustrates some embodiments of the microfluidic devices that can be used in the methods for categorizing and assaying micro-objects.

FIG. 8 depicts some embodiments of the intensity gradients of assay reagents in the chambers of the microfluidic device over time.

FIG. 9 depicts an embodiment of assaying the viability of micro-objects according to the present invention.

FIG. 10 depicts an embodiment of assaying surface markers of micro-objects according to the present inventions.

FIG. 11 compares the results of assaying surface markers of micro-objects using the method of the present invention and FACS.

FIG. 12 illustrates diffusion of assay reagents from a chamber to a flow region (e.g., a microfluidic device) and how an in situ-generated structure affects the diffusion rate.

FIG. 13A shows a fluorescent image at Time 0 after a permeability mixture was introduced into a microfluidic device without an in situ-generated structure formed.

FIG. 13B shows a fluorescent image at Time 60 after a permeability mixture was introduced into a microfluidic device without an in situ-generated structure formed.

FIG. 13C shows a brightfield image showing in situ-generated structures formed and sealed at the opening of each chamber.

FIG. 13D shows a fluorescent image at Time 0 after a permeability mixture was introduced into a microfluidic device having an in situ-generated structure formed at the opening of each chamber.

FIG. 13E shows a fluorescent image at Time 60 after a permeability mixture was introduced into a microfluidic device having an in situ-generated structure formed at the opening of each chamber.

FIG. 14 illustrates changes of diffusion coefficient along with the changes of concentration of hydrogel solution components including polymer component A, polymer component B, crosslinker, initiator, and inhibitors. Top: diffusion coefficient of AF488P-IgG. Bottom: diffusion coefficient of AF647-OVA. In each plot, X-axis indicates the change of concentration of the indicated component in percentage of a standard concentration; Y-axis indicates the diffusion coefficient. Gray area shows the diffusion coefficient measurement within one standard deviation.

FIG. 15A shows an image of six chambers of a microfluidic device. Chambers 1501 and 1504 had a first non-reversible hydrogel barrier 1521 and a reversible hydrogel barrier 1510 formed therewithin. Chambers 1502 and 1505 had a first non-reversible hydrogel barrier 1522 and a reversible hydrogel barrier 1510 formed therewithin. Chambers 1503 and 1506 had only a reversible hydrogel barrier 1510 formed therewithin. FIG. 15A is a brightfield image taken at Time 0 when a dissolution reagent had not yet been introduced.

FIG. 15B shows a brightfield image taken at a time point after a dissolution reagent being introduced and the reversible hydrogel barriers 1510 within chambers 1503 and 1506 were dissolving.

FIG. 15C shows a brightfield image taken at a time point when the reversible hydrogel barriers 1510 within chambers 1501, 1503, 1504, and 1506 were dissolving.

FIG. 15D shows a brightfield image taken at a time point when the reversible hydrogel barriers 1510 within chambers 1503 and 1506 were dissolved and the reversible hydrogel barriers 1510 within chambers 1501 and 1504 were still dissolving.

FIG. 15E shows a brightfield image taken at a time point when the reversible hydrogel barriers 1510 within chambers 1501, 1503, 1504, and 1506 were dissolved and reversible hydrogel barriers 1510 within chambers 1502 and 1505 were still dissolving.

FIG. 15F shows a brightfield image taken at a time point when all the reversible hydrogel barriers 1510 were dissolved.

FIG. 16A illustrates a microfluidic device comprising three rows of chambers, each row having a plurality of chambers disposed along and opening to a corresponding microfluidic channel. A first plurality of in situ-generated structures 1631 block the first channel 1601 and the third channel 1603 so that a first assay reagent 1621 can only enter the second channel 1602. Therefore, the first assay reagent 1621 can only diffuse into the second plurality of chambers 1612 but not the first plurality of chambers 1611 and the third plurality of chambers 1613.

FIG. 16B illustrates the microfluidic device shown in FIG. 16A after the first plurality of in situ-generated structures 1631 have been removed and a second plurality of in situ-generated structures 1632 have been introduced. The second plurality of in situ-generated structures 1632 block the second channel 1602 so that a second assay reagent 1622 can only enter the first channel 1601 and the third channel 1603. Therefore, the second assay reagent 1622 can only diffuse into the first plurality of chambers 1611 and the third plurality of chambers 1613 but not the second plurality of chambers 1612.

FIG. 16C illustrate the microfluidic device shown in FIG. 16B after the second plurality of in situ-generated structures 1632 have been removed. The plurality of chambers 1611 and the third plurality of chambers 1613 retain the second assay reagent 1622 while the second plurality of chambers 1612 retain the first assay reagent 1621.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to a x-axial/z-axial, a y-axial/z-axial, or an x-axial/y-axial area.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one. As used herein, the term “plurality” can be 2, 3, 4, 5, 6, 7, 8, 9, 10, or more.

As used herein: μm means micrometer, μm³ means cubic micrometer, μL means picoliter, nL means nanoliter, and μL (or uL) means microliter.

As used herein, “air” refers to the composition of gases predominating in the atmosphere of the earth. The four most plentiful gases are nitrogen (typically present at a concentration of about 78% by volume, e.g., in a range from about 70-80%), oxygen (typically present at about 20.95% by volume at sea level, e.g. in a range from about 10% to about 25%), argon (typically present at about 1.0% by volume, e.g. in a range from about 0.1% to about 3%), and carbon dioxide (typically present at about 0.04%, e.g., in a range from about 0.01% to about 0.07%). Air may have other trace gases such as methane, nitrous oxide or ozone, trace pollutants and organic materials such as pollen, diesel particulates and the like. Air may include water vapor (typically present at about 0.25% or may be present in a range from about 10 ppm to about 5% by volume). Air may be provided for use in culturing experiments as a filtered, controlled composition and may be conditioned as described herein.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150,200, 250, 300, 400, 500, 600,700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 μL to 1 nL, 100 μL to 2 nL, 100 μL to 5 nL, 250 μL to 2 nL, 250 μL to 5 nL, 250 μL to 10 nL, 500 μL to 5 nL, 500 μL to 10 nL, 500 μL to 15 nL, 750 μL to 10 nL, 750 μL to 15 nL, 750 μL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. Nos. 6,408,878 and 9,227,200, each of which is herein incorporated by reference in its entirety.

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by absorbance of light by objects in the field of view.

As used herein, “structured light” is projected light that is modulated to provide one or more illumination effects. A first illumination effect may be projected light illuminating a portion of a surface of a device without illuminating (or at least minimizing illumination of) an adjacent portion of the surface, e.g., a projected light pattern, as described more fully below, used to activate DEP forces within a DEP substrate. When using structured light patterns to activate DEP forces, the intensity, e.g., variation in duty cycle of a structured light modulator such as a DMD, may be used to change the optical power applied to the light activated DEP actuators, and thus change DEP force without changing the nominal voltage or frequency. Another illumination effect that may be produced by structured light includes projected light that may be corrected for surface irregularities and for irregularities associated with the light projection itself, e.g., fall-off at the edge of an illuminated field. Structured light is typically generated by a structured light modulator, such as a digital mirror device (DMD), a microshutter array system (MSA), a liquid crystal display (LCD), or the like. Illumination of a small area of the surface, e.g., a selected area of interest, with structured light improves the signal-to-noise-ratio (SNR), as illumination of only the selected area of interest reduces stray/scattered light, thereby lowering the dark level of the image. An important aspect of structured light is that it may be changed quickly over time. A light pattern from the structured light modulator, e.g., DMD, may be used to autofocus on difficult targets such as clean mirrors or surfaces that are far out of focus. Using a clean mirror, a number of self-test features may be replicated such as measurement of modulation transfer function and field curvature/tilt, without requiring a more expensive Shack-Hartmann sensor. In another use of structured light patterns, spatial power distribution may be measured at the sample surface with a simple power meter, in place of a camera. Structured light patterns may also be used as a reference feature for optical module/system component alignment as well used as a manual readout for manual focus. Another illumination effect made possible by use of structured light patterns is selective curing, e.g., solidification of hydrogels within the microfluidic device.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, glass beads, amorphous solid substrates, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescent labels, proteins (including receptor molecules), carbohydrates, antigens, small molecule signaling moieties, or other chemical/biological species capable of use in an assay. In some variations, beads/solid substrates including moieties/molecules may be capture beads, e.g., configured to bind molecules including small molecules, peptides, proteins or nucleic acids present in proximity either selectively or nonselectively. In one nonlimiting example, a capture bead may include a nucleic acid sequence configured to bind nucleic acids having a specific nucleic acid sequence or the nucleic acid sequence of the capture bead may be configured to bind a set of nucleic acids having related nucleic acid sequences. Either type of binding may be understood to be selective. Capture beads containing moieties/molecules may bind nonselectively when binding of structurally different but physico-chemically similar molecules is performed, for example, size exclusion beads or zeolites configured to capture molecules of selected size or charge. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g., about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

As referred to herein, “gas permeable” means that the material or structure is permeable to at least one of oxygen, carbon dioxide, or nitrogen. In some embodiments, the gas permeable material or structure is permeable to more than one of oxygen, carbon dioxide and nitrogen and may further be permeable to all three of these gases.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion, and may encompass perfusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result. Flowing can comprise pulling solution through and out of the microfluidic channel (e.g., aspirating) or pushing fluid into and through a microfluidic channel (e.g., perfusing).

The phrase “substantially no flow” refers to a rate of flow of a fluidic medium that, when averaged over time, is less than the rate of diffusion of components of a material (e.g., an analyte of interest) into or within the fluidic medium. The ratio of a rate of flow of a component in a fluidic medium (i.e., advection) divided by the rate of diffusion of such component can be expressed by a dimensionless Peclet number. Thus, a region within a microfluidic device that experiences substantially no flow in one in which the Peclet number is less than 1. The Peclet number associated with a particular region within the microfluidic device can vary with the component or components of the fluidic medium being considered (e.g., the analyte of interest), as the rate of diffusion of a component or components in a fluidic medium can depend on, for example, temperature, the size, mass, and/or shape of the component(s), and the strength of interactions between the component(s) and the fluidic medium. In certain embodiments, the Peclet number associated with a particular region of the microfluidic device and a component located therein can be 0.95 or less, 0.9 or less, 0.85 or less, 0.8 or less, 0.75 or less, 0.7 or less, 0.65 or less, 0.6 or less, 0.55 or less, 0.5 or less, 0.4 or less, 0.3 or less, 0.2 or less, 0.1 or less, 0.05 or less, 0.01 or less, 0.005 or less, or 0.001 or less.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

As used herein, a “flow path” refers to one or more fluidically connected circuit elements (e.g., channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” confines a micro-object to a defined area within the microfluidic device.

As used herein, “pen” or “penning” refers to disposing micro-objects within a chamber (e.g., a sequestration pen) within the microfluidic device. Forces used to pen a micro-object may be any suitable force as described herein such as dielectrophoresis (DEP), e.g., an optically actuated dielectrophoretic force (OEP); gravity; magnetic forces; or tilting. In some embodiments, penning a plurality of micro-objects may reposition substantially all the micro-objects. In some other embodiments, a selected number of the plurality of micro-objects may be penned, and the remainder of the plurality may not be penned. In some embodiments, when selected micro-objects are penned, a DEP force, e.g., an optically actuated DEP force or a magnetic force may be used to reposition the selected micro-objects. Typically, micro-objects may be introduced to a flow region, e.g., a microfluidic channel, of the microfluidic device and introduced into a chamber by penning.

As used herein, “unpen” or “unpenning” refers to repositioning micro-objects from within a chamber, e.g., a sequestration pen, to a new location within a flow region, e.g., a microfluidic channel, of the microfluidic device. Forces used to unpen a micro-object may be any suitable force as described herein such as dielectrophoresis, e.g., an optically actuated dielectrophoretic force; gravity; magnetic forces; or tilting. In some embodiments, unpenning a plurality of micro-objects may reposition substantially all the micro-objects. In some other embodiments, a selected number of the plurality of micro-objects may be unpenned, and the remainder of the plurality may not be unpenned. In some embodiments, when selected micro-objects are unpenned, a DEP force, e.g., an optically actuated DEP force or a magnetic force may be used to reposition the selected micro-objects.

As used herein, “export” or “exporting” refers to repositioning micro-objects from a location within a flow region, e.g., a microfluidic channel, of a microfluidic device to a location outside of the microfluidic device, such as a 96 well plate or other receiving vessel. The orientation of the chamber(s) having an opening to the microfluidic channel permits easy export of micro-objects that have been positioned or repositioned (e.g., unpenned from a chamber) to be disposed within the microfluidic channel. Micro-objects within the microfluidic channel may be exported without requiring disassembly (e.g., removal of the cover of the device) or insertion of a tool into the chamber(s) or microfluidic channel to remove micro-objects for further processing.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

As used herein, a “non-sweeping” rate of fluidic medium flow means a rate of flow sufficient to permit components of a second fluidic medium in an isolation region of the sequestration pen to diffuse into the first fluidic medium in the flow region and/or components of the first fluidic medium to diffuse into the second fluidic medium in the isolation region; and further wherein the first medium does not substantially flow into the isolation region.

Microfluidic devices have been used for performing various kinds of assays in the field. There is a need to assay micro-objects in a simple and instant manner, so that information can be collected for immediate industrial or clinical uses. The present disclosure provides an efficient way to assay a micro-object by retaining a reagent within a microfluidic device before introducing the micro-objects to be assayed and to perform the interaction between the micro-objects and the reagent are driven by diffusion.

Methods of Contacting a Reagent with a Micro-Object.

The present disclosure provides a method of contacting a micro-object with a reagent within a microfluidic device, using a chamber of the microfluidic device as a reservoir for the reagent required to assay the micro-object. The microfluidic device can comprise a microfluidic circuit material defining a flow region and a chamber comprising a proximal opening fluidically connecting the chamber to the flow region. The method can comprise: introducing a micro-object into the flow region of the microfluidic device; and allowing a reagent to diffuse from the chamber to the flow region to contact the micro-object. In some embodiments, the proximal opening of the chamber is oriented substantially parallel to a flow of a fluidic medium in the flow region. In some embodiments, the method further comprises observing an interaction between the reagent and the micro-object.

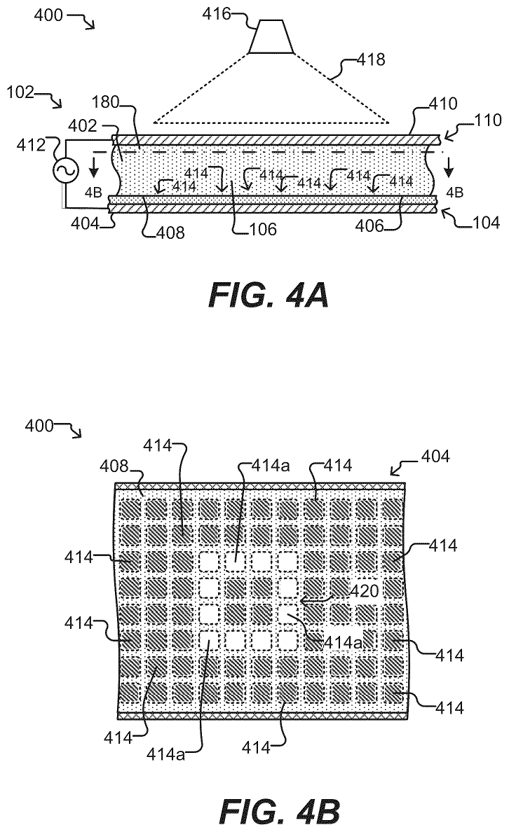

In the embodiment shown in FIG. 6, a first fluidic medium 611 containing a reagent 612 configured to interact with a micro-object 640 can be introduced into a flow region 620 (e.g. a microfluidic channel) of the microfluidic device, as shown in box 601. In this embodiment, the reagent 612 is an AOPI dye (acridine orange (AO) and propidium iodide (PI)). Note that the flow 613 of the first fluidic medium 611 is substantially parallel to an opening 631 of the chamber 630. Over time, the reagent 612 in the first fluidic medium 611 will diffuse from the flow region 620 into the chamber 630 whereby the reagent is present in the chamber 630 (arrow 614 indicates the overall direction of the diffusion of the reagent 612), as shown in box 602.

Next, a second fluidic medium containing the micro-objects 640 (e.g., a cell) can be flowed into the flow region 620 (Box 603). The introduction of the second fluidic medium will displace part or all of the first fluidic medium 611 in the flow region 620 (Box 603). Without wishing being bound by theory, the chamber 630 can be designed to comprise an unswept region so that the reagent 612 can be present or retained within the chamber 630 before the second fluidic medium is perfusing in the flow region.

In some embodiments, the chamber is a sequestration pen as described herein. In certain embodiments, the chamber comprises an isolation region and a connection region fluidically connecting the isolation region to the flow region, wherein the isolation region is an unswept region of the microfluidic device. The unswept region, as defined herein, can be fluidically connected to a swept region of the microfluidic device, provided the fluidic connections are structured to enable diffusion but substantially no flow of media between the swept region and the unswept region. In some embodiments, the first assay reagent is present within the isolation region, but it is not so limited that the first assay reagent can only be present within the isolation region. In some situations, the reagent can also be present within the connection region, or both the connection region and the isolation region.

Once the second fluidic medium with the micro-objects partially or completely fills the flow region, e.g., the desired quantity of micro-objects 640 has been introduced to the flow region, the flow of the second fluidic medium can be stopped. In some instances, some of the micro-objects 640 so introduced may be disposed proximal to the opening 631 of the chamber 630. The reagent 612 can then diffuse from the chamber 630 to the flow region, as shown in box 604 (arrow 615 indicates the overall direction of the diffusion of the reagent 612). Over time, the reagent 612 is allowed to react with the micro-objects 640 within the flow region 620 (Box 604). In this embodiment, the micro-objects 640 can be stained by the reagent 612. Images can then be taken to observe the result of the staining. Based on the observation, the micro-objects can be categorized. The assayed micro-objects 640 can then be exported and collected outside the microfluidic device.

As described herein, “allowing the reagent to diffuse from the chamber to the flow region” or similar description refers to an overall bulk direction of reagent diffusion. Such language does not require that every molecule of the reagent to be diffusing toward the indicated direction at any given time, but instead indicate that, on average, the molecules of the reagent diffuse toward that direction over time. It is also not limited to every molecule of the reagent arriving at the flow region but only indicates the flow region as the general direction of the diffusion.

Diffusion of Reagent and Concentration Equilibration. The reagent is allowed to diffuse between the flow region and the chamber depending on the concentration difference therebetween. In some embodiments, there are at least two diffusion processes taking place in the methods of the present disclosure (Box 602 and Box 604). As shown in Box 602, the diffusion from the flow region 620 into the chamber 630 (Arrow 614 indicates the direction of the diffusion) allows the reagent 612 to enter the chamber 630 and be retained therewithin. On the other hand, as shown in Box 604, the diffusion from the chamber 630 to the flow region 620 (Arrow 615 indicates the direction of the diffusion) allows the retained reagent 612 to diffuse out of the chamber and interact with the micro-objects 640 in the flow region 620.

The time required for the diffusion depends on various parameters including, but not limited, to the size of the reagent (e.g., molecular weight); the dimensions of the flow region (e.g. the microfluidic channel) and the respective chamber(s) connected thereto; and the viscosity of the fluidic medium comprising the reagent. In some embodiments, the diffusion of the reagent from the flow region into the chamber or from the chamber to the flow region is performed for a least about 0.5, 1, 3, 5, 10, 20, 40, 60, 90, 120, 150, 200, or 300 minutes, or any range formed by two of the foregoing end points In some embodiments, the time required for diffusion of the reagent from the flow region into the chamber and from the chamber to the flow region are substantially the same.

In some embodiments, the diffusion of the reagent reaches a substantially uniform concentration across the flow region (e.g. the microfluidic channel) and the chamber. The term “substantially uniform concentration” or the like used herein refers to a concentration of the reagent that is substantially the same in the flow region and in the chamber. In some embodiments, the concentration of the reagent reaches an equilibrated concentration, so that the difference between the concentration in the flow region and in the chamber is insignificant (e.g., the concentration in the flow region and the concentration is substantially the same).

In some embodiments, allowing the reagent to diffuse from the flow region into the chamber comprises allowing the reagent to equilibrate between the flow region and the chamber. In some embodiments, allowing the reagent to diffuse from the chamber to the flow region to contact the micro-object within the flow region comprises allowing the reagent to equilibrate between the flow region and the chamber. However, the method does not require that the concentration of the reagent reaches equilibrium throughout the flow region and chamber. Instead, the method may be performed at any time point when the reagent has reached a concentration that is sufficient to permit a detectable result of the reaction of the reagent and the micro-object to be obtained.

In some embodiments, the substantially uniform concentration may be substantially the same as or lower than an initial concentration of the reagent introduced into the microfluidic device. As mentioned above, there are at least two diffusion processes taking place in the method of the present disclosure. The first diffusion process takes place when the reagent is introduced to the flow region and diffuses from the flow region into the chamber. The first diffusion process can be performed by two modes.

Taking FIG. 6 as an example, in one mode, allowing the reagent to diffuse from the flow region into the chamber comprises maintaining a continuous perfusion of the first fluidic medium comprising the reagent. In certain embodiments, while introducing the reagent, the flow of the first fluidic medium is continued while the reagent diffuses into the chamber until the diffusion process is deemed complete. In this circumstance, when an equilibrium is reached (602 of FIG. 6), the concentration of the reagent will be of a first substantially uniform concentration, which is substantially the same as the initial concentration of the reagent in the first fluidic medium.

In another mode, after introducing the first fluidic medium comprising the reagent, the flow of the first fluidic medium can be stopped at some time point. The time point can be when the first fluidic medium has been introduced and resides in the flow region so that the reagent in the first fluidic medium can effectively diffuse into the chamber. In some embodiments, the time point can be when the first fluidic medium has displaced an initial fluidic medium residing in the flow region. In other embodiments, the time point can be when the first fluidic medium has substantially filled the flow region. Either way, the reagent in the first fluidic medium is allowed to diffuse into the chamber. In this circumstance, when an equilibrium is reached (602 of FIG. 6), the substantially uniform concentration will be lower than the initial concentration of the reagent in the first fluidic medium because the reagent is diluted during diffusion.

In either mode, the concentration of the reagent can be further diluted later when the reagent diffuses from the chamber to the flow region (604 of FIG. 6), after introducing the micro-object into the flow region.

Because the concentration of the reagent will be diluted during various parts of the methods described herein, the initial concentration of the reagent is selected to be high enough to compensate for the dilution so that the final concentration of the reagent after diffusion out of the chamber can be substantially equal to or higher than a working concentration for the reaction. The working concentration refers to a concentration of the reagent that permits an interaction between the reagent and the micro-object. In some embodiments, the working concentration can be predetermined by conducting a preliminary experiment outside the microfluidic device or be predetermined according to a manual if a commercial reagent is used. In some embodiments, the final concentration of the reagent after diffusion out of the chamber is sufficient to react with the micro-object to provide desired outcome.

The initial concentration and the working concentration may have a relationship with a formula of:

$${C}_{inital} \times [ Dilution Factor ] \geq {C}_{w}$$

where Cᵢₙᵢₜᵢₐₗ is an initial concentration of the reagent being introduced into the flow region; C_{w} is a working concentration of the reagent; and the Dilution Factor accounts for the change in concentration as the reagent diffuses between the chamber(s) and the flow region. In some embodiments, the Dilution Factor comprises a Dilution Factor_{CHFR} (from the chamber to the flow region). In other embodiments, the Dilution Factor further comprises a Dilution Factor_{FRCH} (from the flow region into the chamber) if the dilution processes are taking place both when the reagent diffuses into the chamber and out of the chamber.

The Dilution Factor can be a volume ratio of a volume of the chamber to a total volume of the chamber and the flow region. The Dilution Factor can also be a volume ratio of a volume of the flow region to a total volume of the chamber and the flow region (as shown in the following formula). In the embodiments where the microfluidic device comprises a plurality of chambers, a total volume of all chambers is used to calculate the volume ratio. In some embodiments, the volume ratio of a total volume of the chamber to a total volume of the chamber and the flow region is at least about 0.01, about 0.02, about 0.05, about 0.1, about 0.25, about 0.5, or about 0.75, or any range formed by two of the foregoing end points.
Volume ratio #1 (Dilution Factor_{CHFR}): V_{chamber}/Vₜₒₜₐₗ
Volume ratio #2 (Dilution Factor_{FRCH}): V_{channel}/Vₜₒₜₐₗ

In some embodiments, the Dilution Factor can be approximated by considering the width of the microfluidic channel and the length of the chamber. This approximation may be made when the height of the chamber and the height of the flow region (microfluidic channel) is uniform, e.g., substantially the same, throughout the microfluidic device. When the height dimension might be different between the flow region and the chamber, the differences would need to be accounted for to approximate the volumes of the two regions accurately.

In specific embodiments of two dilution processes, the width of the microfluidic channel is about 225 microns, the length of the chamber is about 370 microns, and so the formula will be:

$${C}_{initial} \times 225 / ( 225 + 370 ) \times 370 / ( 225 + 370 ) = {C}_{uniform}$$

where Cᵢₙᵢₜᵢₐₗ is an initial concentration; C_{uniform} is a substantially uniform concentration (i.e., second substantially uniform concentration) after diffusion. The ratio of the initial concentration and the substantially uniform concentration can be calculated as:

$${C}_{initial} / {C}_{uniform} = 59 \; 5 / 370 \times 595 / 225 = 4.253$$

Because the C_{CHFR} should be equal or higher than the working concentration, the initial concentration of the reagent should be at least about 4 times higher than the working concentration in this embodiment so that, after the diffusion, the concentration of the reagent is sufficient to assay the micro-object in the flow region. In some embodiments, the initial concentration can be at least about 1, about 2, about 4, about 8, about 12, about 16, about 20, about 40, about 100, about 200, or about 2000 times, or any range formed by two of the foregoing end points higher than a working concentration of the reagent in the microfluidic device. In other embodiments, the initial concentration can be a range of any of these amounts.

In some embodiments, the second fluidic medium with the micro-objects resides in the channels of the microfluidic device for a time sufficient for the reagent retained in the chambers to diffuse into the second fluidic medium and react with the micro-objects in the flow region near the channel. During this process, the reagent reacts with the micro-objects sufficiently to allow them to be categorized and/or assayed. In some embodiments, the reaction process can take from about 0.5 min, about 1 minute, about 3 minutes, about 5 minutes, about 20 minutes, about 40 minutes, about 1 hour, about 2 hours, or about 5 hours. In other embodiments, the reaction process can be a range of any of these amounts. In some embodiments, the reaction process can be performed for about 18 minutes. Of course, the reaction process can be longer or shorter than these times provided that the reagent has sufficiently reacted with the desired micro-object(s).

Method of sampling a micro-object population. In some embodiments, introducing a micro-object into the flow region of the microfluidic device comprises introducing a plurality of micro-objects into the flow region. In such embodiments, the method comprises observing an interaction between a first assay reagent and the plurality of micro-objects. In some embodiments, observing an interaction between the first assay reagent and the plurality of micro-objects comprises determining a first percentage of the plurality of micro-objects that interact with the first assay reagent.

In some embodiments, the plurality of micro-objects is a subset of a micro-object population (e.g., a sample of a larger micro-object population). The micro-object population might be maintained in a location separated from the microfluidic device of the present disclosure, and a subset of the micro-object population can be collected and introduced into the flow region of the microfluidic device for assaying. Characterizing the plurality of micro-objects can provide information representative of the entire micro-object population. For example, the micro-object population may have the same or similar percentage of micro-objects that interact with the reagent as does the plurality of micro-objects. In this way, the methods of the present disclosure can be used to sample/characterize a micro-object population without the need to assay the entire micro-object population.

The plurality of micro-objects, being a subset of the micro-object population, can contain 5%, 10%, 15%, 20%, 25%, 30%, 35%, 40%, 45%, 50%, or any range formed by two of the foregoing end points of the total micro-objects of the micro-object population. In some embodiments, method of sampling a micro-object population of the present disclosure can be conducted 2, 3, 4, 5, 6, 7, 8, 9, or 10 times on the same micro-object population to gain a representative information of the entire micro-object population.

In some embodiments, introducing the reagent in the first fluidic medium comprises introducing a first assay reagent and a second assay reagent, wherein the first assay reagent and second assay reagent diffuse into and are retained within the chamber. In such embodiments, the method comprises: determining a first percentage of cells of the subset that interact with the first assay reagent; determining a second percentage of cells of the subset that interact with the second reagent; and/or determining a third percentage of cells of the subset that interact with the first assay reagent and the second reagent. The first percentage, the second percentage, and the third percentage can be used to estimate a percentage of cells of the micro-object population that interact with the first assay reagent, the second assay reagent, or both, respectively.

### Microfluidic Device.

The microfluidic device used in the methods of the present disclosure comprises a flow region and a chamber (or chambers) comprising a proximal opening fluidically connecting the chamber to the flow region. In some embodiments, the flow region comprises a microfluidic channel, and the opening of the chamber is oriented substantially parallel to a flow of fluidic medium in the microfluidic channel. Without wishing to be bound by theory, the chamber is configured to retain the reagent therewithin, acting as a reservoir, and subsequently allows the reagent to diffuse out from the chamber into the flow region when needed. In some embodiments, the reagent is retained within the chamber because the chamber is an unswept region of the microfluidic device.

As used herein, “retain” refers to the propensity for the reagent to remain within a space despite the ability for a portion of the reagent to diffuse out of the space. In preferred embodiments, the portion of the reagent remaining in the space is sufficient for its intended use and/or the amount of reagent leaving the space is negligible. In certain embodiments, a portion (e.g., at least 10%, 20%, 30%, 40%, 50%, 60%, 70%, 80%, 90%, 95%, 99%, or more, or any range formed by two of the foregoing end points) of the reagent is retained in the chamber through the process of introducing the micro-object to be assayed into the flow region.

In some embodiments, the microfluidic device comprises a plurality of chambers, each chamber of the plurality comprising the reagent present therewithin. In other words, each of the plurality of chambers can serve as a reservoir storing the reagent needed for assaying a micro-object.

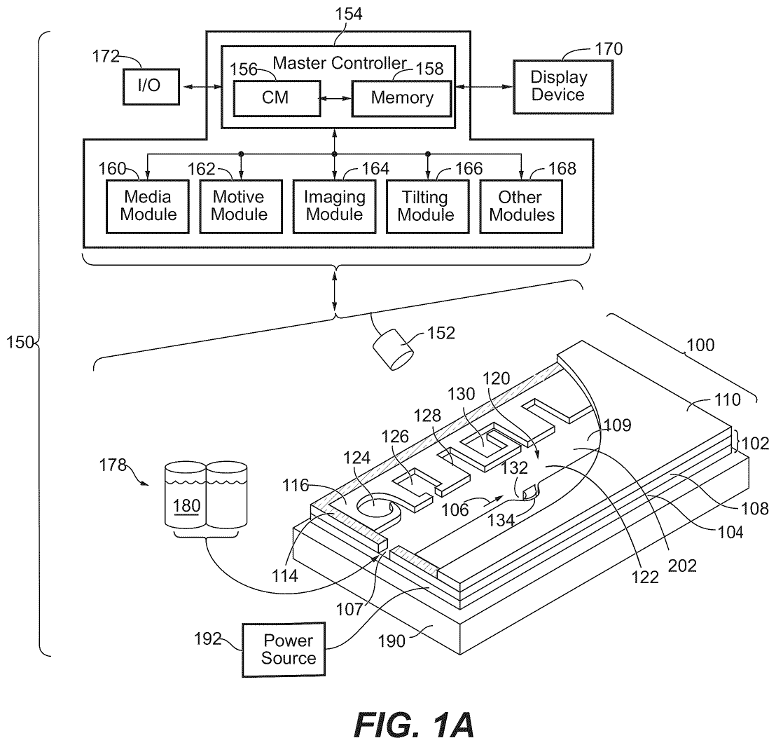

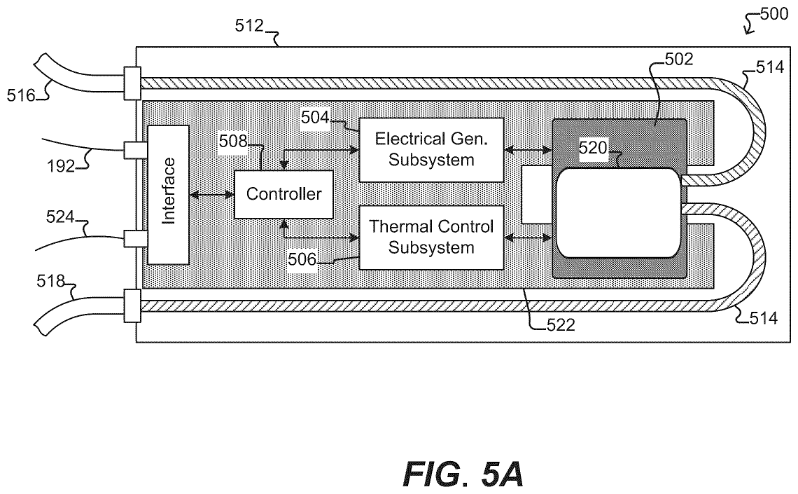

FIG. 7 shows an exemplary configuration of a microfluidic device that can be used in the methods of the present disclosure. The microfluidic device comprises a flow region comprising a plurality of microfluidic channels, which can be optionally arranged in substantially parallel rows (for example, microfluidic channels 711, 712, 713 in FIG. 7). Alternatively, the microfluidic channels of the plurality may be arranged in a serpentine manner (for example, microfluidic channels 122 in FIG. 1B). Each microfluidic channel has a direct fluidic connection to an inlet 701 (see also inlet/outlet 107 in the upper left corner of FIG. 1B) and a direct fluidic connection to an outlet 702 (see also inlet/outlet 107 in the lower right corner of FIG. 1B). The microfluidic device further comprises a plurality of chambers. Each of the plurality of chambers 721 (see also chamber 128 in FIG. 1B) is fluidically connected and has a proximal opening 722 to at least one of the plurality of microfluidic channels.

When a fluidic medium is introduced into the flow region through the inlet 701, the fluidic medium can enter each of microfluidic channels 711, 712, 713 and exit the microfluidic channel from the outlet 702 as indicated by the arrows in the figure (for exemplary purposes, only three arrows are indicated respectively for the entrance and exit of the fluidic medium; see also arrows 106 in FIG. 1B). Any one of the chambers can serve as a reservoir of the reagent. Because all microfluidic channels connect directly to the inlet 701, once the micro-objects are introduced into the microfluidic device, the micro-objects can be quickly distributed among all the microfluidic channels 711, 712, 713. The reagent retained in the chambers 721 is then allowed to diffuse and react with the micro-objects within the channels. This configuration provides a large space for a group of micro-objects to be assayed quickly.

The microfluidic device can be any of the microfluidic devices disclosed herein or otherwise known in the art, with the understanding that the primary requirements of the microfluidic device are a flow region and a chamber fluidically connected to the flow region. In some embodiments, the microfluidic device comprises a transparent cover. In some embodiments, the microfluidic device comprises a transparent a base. In still other embodiments, the microfluidic device comprises a transparent cover and a transparent base.

In some embodiments, the cover and/or base comprises a plastic substrate. In some embodiments, the cover and/or base comprises a glass substrate. In some embodiments, the cover and/or the base comprises a polymer substrate (e.g., a polydimethylsiloxane (PDMS) substrate, or similar polymers known in the art). In some embodiments, the cover or the base comprises a substrate which is opaque. In some embodiments, the cover and/or base comprises a substrate which comprises a semiconductor material and which may be part of a DEP configuration. In other embodiments, the cover and/or the base comprises a substrate that in non-conductive and is not part of a DEP configuration. In some embodiments, the cover comprises a first substrate (e.g., a glass substrate) and the base comprises a second substrate (e.g., a semi-conductor substrate) and the first and second substrates are different from one another.

### Reagent.

The reagents used in the methods of the present disclosure can be selected as desired, based on the assays to be performed on the micro-objects. The reagent can be, but not limited to, a protein, a peptide, a chemical, a glycan, a polymer, or a combination thereof. In some embodiments, the reagent is configured to bind to a micro-object of interest or to be bound by a micro-object of interest with specificity. Preferably, the reagent is flowable or diffusible within the microfluidic device. Preferably, the reagent is soluble, for example, in a fluidic medium used to introduce to the reagent and/or in a fluidic medium residing within the microfluidic device. The size of the reagent can be of a variety of ranges. For example, a reagent used in the methods of the present disclosure can be about 1 kDa, 5 kDa, 10 kDa, 50 kDa, 100 kDa, 200 kDa, 300 kDa, 400 kDa, 500 kDa, 600 kDa, 700 kDa, 800 kDa, 900 kDa, or 1000 kDa, or higher, or any value therebetween.

The regent used in the methods of the present disclosure can be categorized into three types based on their intended uses in the methods. The first type of reagent (i.e., an assay reagent) is used to assay a micro-object of interest (e.g., to assay the micro-object of interest) including facilitating an assay on the micro-object. The type of an assay reagent can varies depending on what assay is going to be performed on the micro-object. The second type of reagent (i.e., a reporter reagent) is used to observe or facilitate the observation of the interaction between the assay reagent and the micro-object. Preferably, a reporter reagent is detectable or is labeled with a detectable label. The third type of reagent is used to form an in situ-generated structure that creates a physical barrier facilitating an assay to be performed. In some embodiment, the third type of reagent can comprise a prepolymer composition, preferably, a flowable prepolymer composition, configured to form an in situ-generated structure at a selected area within the microfluidic device. The in situ-generated structure can facilitate assaying the micro-object, which is described in further details below.

In some embodiments, a reagent can serve both the intended purpose of the assay reagent and the reporter reagent, for example, the assay reagent can be a detectable reagent. In certain embodiments, an assay reagent can be a fluorescent dye configured to stain a micro-object of interest. In these embodiments, the assay reagent not only is used to assay the micro-object interest but also can be detected to indicate the interaction between the assay reagent and the micro-object. Therefore, observing an interaction between the assay reagent and the micro-object can comprise detecting a signal associated with the assay reagent, and there is no need for a reporter reagent.

In some embodiments, the interaction between the assay reagent and the micro-object can be observed without the aid of a reporter reagent or any other detectable reagents. In certain embodiments, observing an interaction between the assay reagent and the micro-object comprises observing a morphologic change of the micro-object. The morphologic change can be observed using a microscope under brightfield. Therefore, there is no need for a reporter reagent or any other detectable reagents.

In certain embodiments where both an assay reagent and a reporter reagent are used, the assay reagent and the reporter reagent can be introduced into the flow region together, for instance, by introducing a fluidic medium comprising both the assay reagent and the report reagent into the flow region and allowing them to diffuse into the chamber as described above. In some other embodiments, the assay reagent and the report reagent can be introduced separately. In some embodiments, the report reagent can be introduced together with the micro-object.

The assay reagent and the reporter reagent can have different molecular weights thereby having different diffusion rates. In certain embodiments, the reporter reagent diffuses faster than the assay reagent does, while in some other embodiments, the reporter reagent diffuses slower than the assay reagent does. Alternatively, the diffusion rates of the assay reagent and the reporter reagent are substantially the same. It is noted that the diffusion rate described herein refers to the diffusion taking place within the microfluidic environment where the method of the present disclosure is performed.

In some embodiments, the reagent is a mixture of assay reagents or a mixture of an assay reagent, a reporter reagent, and/or a prepolymer composition as described herein. In some embodiments, a first fluidic medium comprising a first assay reagent and a second assay reagent is introduced, wherein the first assay reagent and the second assay reagent are different kinds of assay reagents. In such embodiments, the method comprises observing an interaction between the micro-object with the first assay reagent and an interaction between the micro-object with the second assay reagent. In some embodiments, more than one kind of reagent is used in the methods, for example, 2, 3, 4, 5, 6, 7, 8, 9, 10 or more kinds of reagents whereby, after diffusion, there are 2, 3, 4, 5, 6, 7, 8, 9, 10 or more kinds of reagents present in the chamber. The more than one kind of reagent can be introduced separately. In some embodiments where more than one kind of assay reagent is used, each kind of the assay reagent can be detectable and spectrally distinct.

As used herein, “different kind of reagents” refers to those reagents that are different in terms of binding site, binding affinity, or both to a micro-object to be assayed. In addition, those assay reagents can be different in terms of their concentrations (i.e., their initial concentration or their substantially uniform concentrations after the diffusion processes) so that interaction of the micro-object with a same type of assay reagent but of different concentrations can be observed.

### Micro-Objects.

The micro-object(s) of the present disclosure can be any kind of object to be assayed. Generally, in various embodiments, the micro-object is insoluble, for example, in a fluidic medium used to introduce to the micro-object and/or in a fluidic medium residing within the microfluidic device. In some embodiments, the micro-object can be a biological micro-object including but not limited to a cell, a protein, a peptide, a nucleic acid, or a combination thereof. In some embodiments, the cell can include but is not limited to an animal cell, a plant cell, a fungus cell, an insect cell, or a bacteria cell. In some embodiments, the cell can be a naturally occurring cell or a genetically engineered cell. Some unlimited examples include tumor cells, T cells (e.g., Pen T cells), CAR-T cells, NK cells, or CAR-NK cells. The micro-object can also be a micro-object produced or released from a cell, for example, a cytokine. The nucleic acid can be a mRNA released from lysing a cell. In some embodiments, the micro-object can be introduced while being carried in a carrier. In such embodiments, allowing the first assay reagent to diffuse from the chamber to the flow region to contact the micro-object comprises releasing the micro-object from the carrier. In some other embodiments, the micro-object can be a bead carrying a biological molecule, comprising but not limited to a protein, a peptide, a nucleic acid, or a combination thereof.

In some embodiments, introducing the micro-object comprises introducing a second fluidic medium comprising the micro-object, and stopping a flow of the second fluidic medium after the micro-object is within the flow region. In some embodiments, introducing a micro-object into the flow region of the microfluidic device comprises introducing a plurality of micro-objects into the flow region. Preferably, introducing the micro-object comprises introducing the micro-object to a position within the flow region that is proximal to the opening of the chamber. In some other embodiments, the micro-object is retained within the flow region (e.g., a microfluidic channel) while the reagent is allowed to diffuse out of the chamber. In such embodiments, allowing the reagent to diffuse from the chamber to the flow region to contact the micro-object comprises contacting the micro-object with the reagent within the flow region.

It is however not limited where the micro-object is positioned when contacting the reagent. While it is preferable that the micro-object contacts the reagent within the flow region, the micro-object might enter the chamber while the method of the present disclosure is performed.

In certain embodiments, the micro-object substantially has no contact with the reagent until the reagent diffuses from the chamber to the flow region and to contact the micro-object. As used herein, “substantially has no contact” refers to a situation where a micro-object might have no contact or minimal contact with the reagent before the regent diffuses out from the chamber. For example, after a reagent is introduced into the flow region and allowed to diffuse into the chamber, a fluidic medium can be introduced to flush out the reagent remaining in the flow region. Therefore, when the micro-object is introduced, there will be no or substantially no reagent within the flow region. The introduced micro-object will substantially have no contact with the reagent until the reagent retained within the chamber diffuses from the chamber into the flow region. Nevertheless, it is not limited that in a rare situation, there might be reagent remaining within the flow region and contact with the micro-object while the majority of the reagent is still retained, has not yet diffused out, within the chamber provided that the contact is not intentional for performing the method of the present disclosure.

In some embodiments, the micro-object is introduced together with a reporter reagent. In such embodiments, a micro-object can mix with a reporter reagent and be introduced together in a fluidic medium. Alternatively, a fluidic medium comprising a micro-object and a fluidic medium comprising a reporter reagent are introduced into the flow region simultaneously so that the micro-object and the reporter reagent mix with each other within the flow region while being introduced.

### In Situ-Generated Structure.

In many embodiments of the present disclosure, an in situ-generated structure can facilitate assaying a micro-object of interest in the methods of the present disclosure. As used herein, an “in situ-generated structure” refers to a structure that is formed in a selected area while the microfluidic device is in operation. The structure is not formed while manufacturing the microfluidic device or does not exist before the microfluidic device is used for experiments or research. The term “structure” refers to a physical structure that is formed and fixed, at least for a certain period of time, in a selected area and is capable of impeding or blocking passage of a particle (e.g., a micro-object or a molecule) through the structure. The in situ-generated structure will be described in detail thereafter and can be those described in U.S. Patent Application Publication No. 20170165667, filed on Nov. 22, 2016, which is incorporated by reference herein in their entirety.

The in situ-generated structure may include a solidified polymer network, which may include a photoinitiated polymer. The solidified polymer network may have at least a portion formed from a photoinitiated polymer. In some embodiments, all of the solidified polymer network may be formed from a photoinitiated polymer. In other embodiments, the solidified polymer network may have at least a portion formed from a thermosensitive polymer. In some embodiments, the polymer of the solidified polymer network may be a synthetic polymer, a modified synthetic polymer, or a biological polymer. The biological polymer may be light or thermally activatable. The synthetic polymer modifications may include size modification motifs, cleavage motifs, or cell recognition motifs. In some embodiments, the polymer may include a modified polyethylene glycol. In some embodiments, the solidified polymer network does not include a silicone polymer. In some embodiments, the solidified polymer network does not include silicon.

The in situ-generated structure may be configured to substantially restrict passage of micro-objects into, out of, and/or through the at least one in situ-generated structure in a size dependent manner. In some embodiments, the in situ-generated structure has a porosity that restricts (or impedes) passage of a molecule within the microfluidic device in a size-dependent manner. As used herein, “restriction (or impede)” refers to affecting the passage of a molecule but does not necessarily block the passage thereof. In some embodiments, the porosity can discriminate between molecules of different sizes. For example, the in situ-generated structure can be porous to an assay reagent but non-porous (i.e., block passage of) to a micro-object. In some embodiments, the in situ-generated structure can be non-porous to both an assay reagent and a micro-object. In some embodiments, the in situ-generated structure can be porous to a first reagent, which may be an assay reagent while being not porous to a second reagent, which may be an assay reagent or a reagent having a different function.

In some embodiments, the solidified polymer network may be configured to be porous to a flow of fluidic medium. In some embodiments, the solidified polymer network is substantially non-porous to a micro-object having a diameter of greater than about 500 nm, 600 nm, 700 nm, 800 nm, 900 nm, 1 micron, 2 microns, 3 microns, 4 microns, 5 microns, 6 microns, 7 microns, 8 microns, 9 microns, 10 microns, 11 microns, 12 microns, 13 microns, 14 microns, 15 microns, or more.

In some embodiments, more than one in situ-generated structure may be generated in the microfluidic device. When more than one in situ-generated structure is generated in a microfluidic device, there may be more than one kind of in situ-generated structure generated, and in any combination.

The in situ-generated structure may be designed to be temporary or it may be kept in place until the conclusion of the experiment/assay/sorting/cloning process being performed in the microfluidic device. The solidified polymer network of the in situ-generated structure may be at least partially removable by application of increased fluid flow through the flow region, hydrolysis, proteolysis, osmotic change, temperature change, optical illumination, or introduction of a chemical cleavage reagent configured to specifically cleave a moiety of the solidified polymer network. In some embodiments, at least a portion of the in situ-generated structure may be removable using a flow of a fluidic medium in the flow region, for one non-limiting example.

Location of the in situ-generated structure. The in situ-generated structure can be formed within a flow region (e.g., a microfluidic channel) or within a chamber (e.g., a sequestration pen) of a microfluidic device. In certain embodiments, the in situ-generated structure can be located in an area proximal to the proximal opening of the chamber to the flow region. For example, the area can be at the edge of the proximal opening of the chamber. Alternatively, the in situ-generated structure can be located in a mid-area of the chamber with respect to an axis of the chamber, for example, with respect to an axis of the chamber that is substantially vertical to a direction of intended flow of medium within the flow region (e.g., a barrier that bisects the chamber into a proximal region and a distal region relative to the opening to the flow region).

In some embodiments, the in situ-generated structure is located within the chamber and extends into the flow region. A portion of the in situ-generated structure may extend from within the connection region into the microfluidic channel. In some embodiments, the portion of the in situ-generated structure extending into the microfluidic channel comprises less than about 50%, about 40%, about 30%, about 20%, about 10% or about 5% of a volume of the in situ-generated structure.

In some embodiments, a width of the in situ-generated structure is about ¼ to about ¾, about ¼ to about ½, or about ¼ to about ⅝ of a width of the chamber, particularly, a width of the connection region or a width of the isolation region. A width of the in situ-generated structure across the isolation region or the connection region may be about 3 microns to about 50 microns, about 5 microns to about 40 microns, about 5 microns to about 30 microns, about 5 microns to about 20 microns, or about 7 microns to about 25 microns. A width of the isolation region or the connection or the isolation region may be about 30 microns to about 50 microns, about 20 microns to about 40 microns, about 30 microns to about 60 microns, about 30 microns to about 90 microns, about 30 microns to about 120 microns, or about 30 microns to about 250 microns.

The in situ-generated structure may be disposed within the connection region of the chamber and having a dimension across a width of the connection region sized to restrict passage of a molecule. For example, the in situ-generated structure can be an in situ-generated cap sealing the proximal opening of the chamber. As used herein, “seal” refers to that the in situ-generated structure fully encloses the opening of the chamber to the flow region. In some embodiments, the in situ-generated structure is an in situ-generated cap having a dimension across a width of the connection region or a width of the opening of the chamber. However, sealing does not necessarily mean that all kinds of molecules will be blocked from passing through the in situ-generated structure. Whether a molecule is restricted, blocked, or allowed to pass the in situ-generated cap is dependent on the porosity (i.e., pore size) of the in situ-generated structure, that is, in a size-dependent manner.

Formation of the in situ-generated structure. In order to form an in situ-generated structure within the microfluidic device, the method further comprises introducing a prepolymer composition (e.g., a flowable prepolymer composition, and preferably a soluble prepolymer composition, e.g., soluble to a fluidic medium used to introduce the prepolymer composition, which may include more than one component) into the flow region. The prepolymer composition can be allowed to diffuse into the chamber of the microfluidic device. In some embodiments, the method further comprises activating solidification of the prepolymer composition thereby forming the in situ-generated structure within the microfluidic device. Forming the in situ-generated structure can comprise forming a solidified polymer network, which can be formed by photoactivation, temperature change, or osmotic change. In some embodiments, the prepolymer composition is introduced before, after, or together with introducing the assay reagent.

Adjusting diffusion rate of the assay reagent. The in situ-generated structure can have a porosity that restricts the passage of a molecule, for example, the assay reagent of the methods of the present disclosure, so that the diffusion of the molecule passing through the in situ-generated structure is impeded but not blocked. In this way, the diffusion rate of the molecule can be adjusted by tuning the porosity of the in situ-generated structure. As used herein, “diffusion rate” has the same definition as “rate of diffusion” described herein.

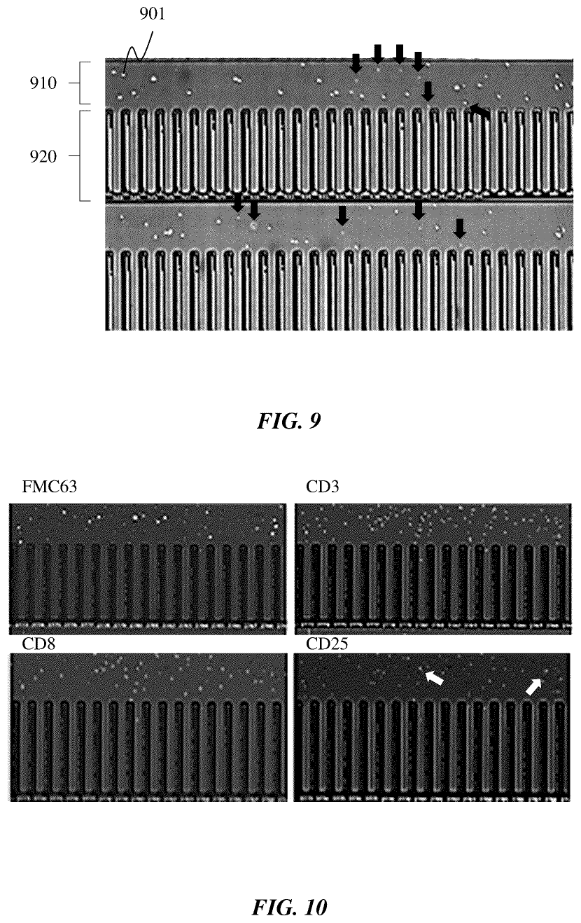

For example, please see FIG. 12, a microfluidic channel 1201 and three chambers 1211, 1212, 1213 are depicted. Each of the three chambers 1211, 1212, 1213 comprises a proximal opening to the microfluidic channel 1201. FIG. 12 illustrates a moment that assay reagents 1220 retained in the chambers 1211, 1212, 1213 are diffusing toward the microfluidic channel 1201. Among those three chambers, an in situ-generated structure 1231 is formed at mid-chamber of chamber 1212, and an in situ-generated structure 1232 is formed in an area proximal to the opening of chamber 1213. The in situ-generated structure 1231 and the in situ-generated structure 1232 both have a porosity that impedes but does not block passage of the assay reagent 1220. As a result, the molecule of interest 2620 is diffusing more slowly out from a region of chamber 2612 distal to in situ-generated structure 2631 and out from a region of chamber 2613 distal to in situ-generated structure 2632 compared to the rate of diffusion of molecule of interest 2620 in chamber 2611.

In certain embodiments, the microfluidic device comprises a first chamber and a second chamber. The method can comprise introducing a first prepolymer composition before introducing any assay reagent into the flow region and forming a first in situ-generated cap to seal the second chamber. The first in situ-generated cap has a porosity that blocks passage of a first assay reagent. Then, a first assay reagent is introduced and allowed to diffuse into the first chamber while blocked from entering the second chamber because the second chamber is sealed by the first in situ-generated cap.

The remaining first assay reagent can then be flushed out, and a second prepolymer composition is introduced into the flow region. A second in situ-generated cap can be formed to seal the first chamber. The second in situ-generated cap has a porosity that blocks passage of the first assay reagent and a second assay reagent. Then, the second chamber is unsealed, and the second assay reagent is introduced and allowed diffuse into the second chamber. In this way, two different kinds of assay reagents are present respectively in two chambers of the microfluidic device. A micro-object can then be introduced after the first chamber and/or the second chamber is unsealed by the first in situ-generated cap and the second in situ-generated cap respectively, meaning the first assay reagent and/or the second assay reagent are allowed to diffuse out of the chamber.

In some embodiments, unsealing the chamber refers to removing the in situ-generated cap and/or reducing the size of the in situ-generated structure (e.g., in situ-generated cap). In some embodiments, the in situ-generated structure may be configured to have a first state and a second state, wherein when the in situ-generated structure is in the first state, it is configured to restrict or block passage of a molecule, and when the in situ-generated structure is in the second state it is configured to permit passage of the molecule. A size of the in situ-generated structure may be reduced by temperature change or optical illumination sufficiently to permit the passage of the molecule. In some embodiments, the size of one of the dimensions of the in situ-generated structure may be configured to be sufficiently reducible to permit passage of the molecule. A size of the in situ-generated structure can be reducible upon application of increased fluid flow through the flow region, hydrolysis, proteolysis, osmotic change, temperature change, optical illumination, or introduction of a chemical cleavage reagent configured to specifically cleave a moiety of the solidified polymer network. Further details regarding transition between the first state and the second state and using a swellable polymer are described in U.S. Patent Application Publication No. 20170165667, filed on Nov. 22, 2016, which is incorporated by reference herein in their entirety.

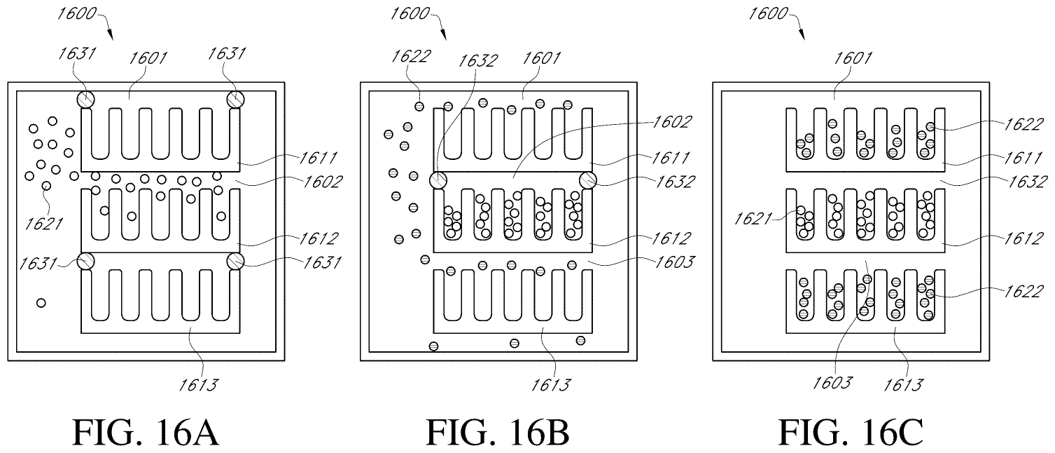

In some embodiments, a microfluidic device 1600 comprises a first plurality of chambers 1611 disposed along and opening to a first channel 1601, a second plurality of chambers 1612 disposed along and opening to a second channel 1602, and a third plurality of chamber 1613 disposed along and opening to a third channel 1603 (FIG. 16A). A first plurality of in situ-generated structures 1631 may be located at a distal edge of the proximal opening of both the first and last chambers of the first plurality of chambers 1611 and at a distal edge of the proximal opening of both the first and last chambers of the third plurality of chambers 1613. The first plurality of in situ-generated structures 1631 may be non-porous, blocking entry of a first assay reagent 1621 to the entire first channel 1601 and the third channel 1603. Therefore, the first assay reagent 1621 will only enter the second channel 1602, thus allowing the first assay reagent 1621 to diffuse into the second plurality of chambers 1612 but not the first plurality of chambers 1611 and the third plurality of chambers 1613.

Turning to FIG. 16B, a second plurality of in situ-generated structures 1632 can be introduced at a distal edge of the proximal opening of both the first and last chambers of the second plurality of chambers 1612. The second in situ-generated structure 1632 may be non-porous, blocking entry of a second assay reagent 1622 to the entire second channel 1602. Therefore, after removing the first plurality of in situ-generated structures 1631, the second assay reagent 1622 can only enter the first channel 1601 and the third channel 1603, thus allowing the second assay reagent 1622 to diffuse into the first plurality of chambers 1611 and the third plurality of chambers 1613 but not the second plurality of chambers 1612. As a result, the second plurality of chambers 1612 can retain assay reagents different from the assay reagents retained within the first plurality of chambers 1611 and the third plurality of chambers 1613 (FIG. 16C). Many other configurations are possible to use the in situ-generated barriers as mechanisms to direct flow, including sample flows containing micro-objects, within the flow region or a channel of a microfluidic device.

### Hydrogel Composition.

In certain embodiments, the in situ-generated structure is a hydrogel. In certain embodiments, the in situ-generated barrier comprises a solidified polymer network. In some embodiments, the solidified polymer network comprises a synthetic polymer, a modified synthetic polymer, or a biological polymer. In certain embodiments, the solidified polymer network comprises at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. In some embodiments, the solidified polymer network does not include a silicone polymer.

Physical and chemical characteristics determining suitability of a polymer for use in the solidified polymer network may include molecular weight, hydrophobicity, solubility, rate of diffusion, viscosity (e.g., of the medium), excitation and/or emission range (e.g., of fluorescent reagents immobilized therein), known background fluorescence, characteristics influencing polymerization, and pore size of a solidified polymer network. The solidified polymer network is formed upon polymerization or thermal gelling of a flowable polymer solution containing at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. Various co-polymer classes may be used, including but not limited to any of the above listed polymers, or biological polymers such as fibronectin, collagen or laminin. Polysaccharides such as dextran or modified collagens may be used. The flowable polymer may be referred alternatively here as a pre-polymer, in the sense that the flowable polymer is crosslinked in-situ. Biological polymers having photoactivatable functionalities for polymerization may also be used.

In some instances, a polymer may include a cleavage motif. A cleavage motif may include a peptide sequence inserted into the polymer that is a substrate for one or more proteases, including but not limited to a matrix metalloproteinase, a collagenase, or a serine proteinase such as Proteinase K. Another category of cleavage motif may include a photocleavable motif such as a nitrobenzyl photocleavable linker which may be inserted into selected locations of the prepolymer. In some embodiments, a nitrobenzyl photocleavable linker may include a 1-methinyl, 2-nitrobenzyl moiety configured to be photocleavable. In other embodiments, the photocleavable linker may include a benzoin moiety, a 1,3 nitrophenolyl moiety, a coumarin-4-ylmethyl moiety or a 1-hydroxy 2-cinnamoyl moiety. A cleavage motif may be utilized to remove the solidified polymer network of an isolation structure. In other embodiments, the polymer may include cell recognition motifs including but not limited to a RGD peptide motif, which is recognized by integrins. Additional cleavable moieties useful for in situ-generated structures are described below.

Polyethylene glycol moieties linked to acrylamide reactive moiety. One type of polymer, amongst the many polymers that may be used, is polyethylene glycol diacrylate (PEGDA) or polyethylene glycol acrylamide (diacrylamide, multi-armed acrylamide or substituted versions as described herein). In a specific example of forming an in situ-generated structure, a solution containing 10% w/v PEGDA (5Kd) and 1% photoinitiator (IRGACURE 2959, 200 Da) was introduced into the device. After allowing equilibration for less than 10 min, the desired region was illuminated with UV light at approximately 340 nm (+/−20 nm), having a power of 400 mW/cm², for 1 second, to initiate polymerization creating the in situ-generated structure.

Polyethylene glycol molecules linked to a norbornene reactive moiety. In other embodiments, a polyethylene glycol polymer molecule including a polyethylene glycol moiety covalently linked to a norbornene reactive moiety may be used as part of a prepolymer, e.g., flowable polymer, composition to be solidified in-situ within a microfluidic device. A first polyethylene glycol polymer molecule will be linked to a crosslinker, and the crosslinker may further be linked to a second polyethylene glycol molecule via a norbornene reactive moiety linked to a second polyethylene glycol moiety of the second polyethylene glycol molecule. The norbornene containing polyethylene glycol molecules react with crosslinkers including thiols, upon photoinitiation in the presence of an initiator to produce thiol-ene crosslinked hydrogels in-situ.

The composition may include: a first and a second polyethylene glycol polymer molecule, each comprising a respective polyethylene glycol moiety and a covalently linked reactive moiety Rₓ; a crosslinker molecule comprising a first reactive moiety R_{xP} disposed at a first end of a linker L moiety and a second reactive moiety R_{xP} disposed at a second end of the linker L moiety, wherein each of the first and the second crosslinker moiety R_{xP} is configured to be activatable to react with the respective reactive moiety Rₓ of the first and the second polyethylene polymer molecules, wherein the first and the second polyethylene polymer molecule comprises different polyethylene glycol moieties or each comprises a same polyethylene glycol moiety.

The first reactive moiety R_{xP} and the second reactive moiety R_{xP} of the first and the second polyethylene glycol molecule may be a norbornenyl moiety. In some embodiments, the norbornenyl moiety may be a substituted norbornenyl moiety. In some embodiments, the substituted norbornenyl moiety may have a hydrophilic substituent, such as but not limited to hydroxy or carboxylate. The substituent does not inhibit the reactivity of the norbornenyl moiety. The norbornenyl moiety is configured to react with a thiol moiety of a crosslinker molecule to form the hydrogel.

The first polyethylene glycol moiety and/or the second polyethylene glycol moiety may include a 1-arm, 2-arm, 4-arm or 8-arm polyethylene glycol moiety. In some embodiments, each arm of the first polyethylene glycol moiety and/or the second polyethylene glycol moiety may include the covalently linked reactive moiety Rₓ. In other embodiments, when the first polyethylene glycol moiety and/or the second polyethylene glycol moiety has a 2-arm, 4-arm, or 8-arm polyethylene moiety structure, at least one arm of the first polyethylene glycol moiety and/or the second polyethylene glycol moiety may include the covalently linked reactive moiety Rₓ and at least one arm of the first polyethylene glycol moiety does not have a covalently linked reactive moiety Rₓ. For example, an 8-arm polyethylene glycol moiety may have seven arms including a reactive moiety Rₓ and one arm that does not include the reactive moiety Rₓ. In some embodiments, an 8-arm polyethylene glycol moiety may have 6-arms, 5-arms, 4-arms, 3-arms, or 2-arms having the reactive moiety Rₓ while the other arms do not have the reactive moiety Rₓ.

The first polyethylene glycol moiety and/or the second polyethylene glycol moiety may have a molecular weight from about 500 Da to about 40 KDa; from about 1 KDa to about 25 KDa; from about 5 KDa to about 25 KDa; from about 5 KDa to about 20 KDa; about 5 KDa to about 15 KDa; or about 5 KDa to about 10 KDa. The second polyethylene glycol moiety may have a molecular weight from about 500 Da to about 40 KDa; from about 1 KDa to about 25 KDa; from about 5 KDa to about 25 KDa; from about 5 KDa to about 20 KDa; about 5 KDa to about 15 KDa; or about 5 KDa to about 10 KDa.

In some embodiments, the first polyethylene glycol moiety may have a molecular weight of about 10 KDa and the second polyethylene glycol moiety may have a molecular weight of about 10 KDa. In other embodiments, the first polyethylene glycol moiety may have a molecular weight of about 10 KDa and the second polyethylene glycol moiety may have a molecular weight of about 20 KDa. When the first polyethylene glycol moiety has a different molecular weight from that of the second polyethylene glycol moiety, the hydrogel once formed will include a mixture of molecules, e.g., some hydrogel molecules will have a 10 KDa polyethylene glycol moiety linked through the crosslinker to a second 10 KDa polyethylene glycol moiety; some hydrogel molecules will have a 10 KDa polyethylene glycol moiety linked through the crosslinker to a 20 KDa polyethylene glycol moiety; and some hydrogel molecules will have a 20 KDa polyethylene glycol moiety linked through the crosslinker to a second 20 KDa polyethylene glycol moiety.

In some embodiments, the first polyethylene glycol molecule and the second polyethylene molecule may be present in the composition in a ratio from about 1:100 to about 100:1. In some further embodiments, the ratio of the first polyethylene glycol molecule and the second polyethylene molecule may be from about 1:1; about 1:2; about 1:3; about 1:4; about 1:5; about 1:6; about 1:7; about 1:8; about 1:9; about 1:10; about 10:1; about 9:1; about 8:1; about 7:1; about 6:1; about 5:1; about 4:1; about 3:1; or about 2:1.

Crosslinker molecule. For polymers including polyethylene glycol moieties linked to a norborenyl moiety as described herein, the crosslinker molecule comprises a first thiol at a first end of the crosslinker molecule and a second thiol moiety at a second end of the molecule, where the first thiol moiety and the second thiol moiety are each configured to reach with a norbornenyl group of either the first or the second polyethylene glycol molecule including a reactive moiety, where the reactive moiety may be a norbornenyl moiety.

In some embodiments, the crosslinker molecule may include a vicinal-diol moiety. A vicinal diol (vic-diol) moiety of the crosslinker provides an in-situ generated hydrogel that may be reversed, e.g, removed or dissolved at a later timepoint, by contact with a periodate (Sodium Periodate), which is a first example of a chemical cleavage reagent that specifically cleaves a polymer including the vic-diol containing crosslinker. This class of hydrogels are typically referred to herein as a reversible hydrogel.

Vic-diol containing crosslinker. In some embodiments of a crosslinker having a vic-diol moiety, the crosslinker has a structure having a molecular formula of Formula I:

HS-LB-CH₂—C(H)(OH)—C(H)(OH)—CH₂-LB-SH Formula I;

where each instance of linker backbone LB is independently selected to comprise 0 to 200 non-hydrogen atoms selected from any combination of silicon, carbon, nitrogen, oxygen, sulfur and phosphorus atoms. In some embodiments, the linker backbone LB may have a linear backbone having carbon atoms. In some embodiments, the linear backbone having carbon atoms does not include any silicon, nitrogen, oxygen, sulfur or phosphorus atoms. When referring to the linear backbone LB having carbon atoms, this does not exclude substitution of hydrogen atom substituents of the carbon backbone with other types of moieties containing alcohols, sulfonates, carboxylic acids and the like. In some embodiments, the crosslinker molecule is dithiothreitol.

Peptide sequence containing crosslinker. In other embodiments, the crosslinker molecule includes a peptide sequence configured to be a substrate to a proteolytic enzyme. The crosslinker containing a peptide sequence that is configured to be cleaved upon contact with a proteolytic enzyme provides an in situ-generated hydrogel that may be reversed, e.g, removed or dissolved at a later timepoint, by contact with a proteolytic enzyme such as, but not limited to a tryptase such as TrypLE™.

In some embodiments of a crosslinker having a peptide moiety, the crosslinker has a structure having a molecular formula of Formula II:

HS-PEPT-SH Formula II

where PEPT is a peptidyl moiety comprising about 4 to about 12 amino acids, and additionally where the peptidyl moiety is susceptible to proteolytic enzymatic cleavage. In some embodiments, the peptide sequence may include GCRDLPRTGGDRCG.

Nonreversible crosslinker. In other embodiments, the crosslinker molecule has a structure of Formula III:

HS-LB4-SH Formula III;

where linker backbone LB4 comprises 3 to 200 non-hydrogen atoms selected from any combination of silicon, carbon, nitrogen, oxygen, sulfur and phosphorus atoms. In some embodiments, the linker backbone LB4 may have a linear backbone having carbon atoms. In some embodiments, the linear backbone having carbon atoms does not include any silicon, nitrogen, oxygen, sulfur or phosphorus atoms. When referring to the linear backbone LB4 having carbon atoms, this does not exclude substitution of hydrogen atom substituents of the carbon backbone with other types of moieties containing alcohols, sulfonates, carboxylic acids and the like. A crosslinker of Formula III provides a hydrogel that is non-reversible, e.g, not dissolvable. In some embodiments, the crosslinker is Sodium 2,3-dimercaptopropanesulfonate monohydrate.

Inhibitor. In some embodiments, the compositions may further include an inhibitor configured to inhibit reaction of the crosslinker with the reactive moiety of the first and/or the second polyethylene glycol polymer molecule and/or of the reactive moiety of the first and/or the second polyethylene glycol polymer molecules upon standing in solution. The inhibitor may be sodium ascorbate, MEHQ, or 4-hydroxy TEMPO. The composition including the first and the second polyethylene glycol polymer molecule, and the crosslinker molecule may further include an inhibitor. In some embodiments, the inhibitor is present within the composition at a concentration from about 0.5 millimolar to about 1.5 millimolar. In other embodiments, the composition may include another component having a photoinitiator and a second amount of the inhibitor. The inhibitor in the component having the photoinitiator may have the inhibitor present in a range from about 5 millimolar to about 20 millimolar. The composition component containing the photoinitiator and second amount of inhibitor may be separately packaged and not combined with the compositions including the first and the second polyethylene glycol polymer molecules until the compositions are introduced into the microfluidic device.

Photoactivated polymerization may be accomplished using a free radical initiator Igracure® 2959 (BASF), a highly efficient, non-yellowing radical, alpha hydroxy ketone photoinitiator, is typically used for initiation at wavelengths in the UV region (e.g., 365 nm), but other initiators may be used. An example of another useful photoinitiator class for polymerization reactions is the group of lithium acyl phosphinate salts, of which lithium phenyl 2,4,6-trimethylbenzolylphosphinate (LAP) has particular utility due to its more efficient absorption at longer wavelengths (e.g., 405 nm) than that of the alpha hydroxy ketone class. Another initiator that may be used are water soluble azo initiators, such as 2,2-Azobis [2-methyl-N-(2-hydroxyethyl)propionamide]. The initiator may be present within the flowable polymer solution at a concentration of about 5 millimolar, about 8 millimolar, about 10 millimolar, about 12 millimolar, about 15 millimolar, about 18 millimolar, about 20 millimolar, about 22 millimolar, about 25 millimolar, about 28 millimolar, about 30 millimolar, about 35 millimolar, or about 40 millimolar.

Crosslinking may be performed by photopatterning of linear or branched PEG polymers, free radical polymerization of PEG acrylates or PEG acrylamides, and specifically tailored chemical reactions such as Michael addition, condensation, Click chemistry, native chemical ligation and/or enzymatic reactions. In particular, photopatterning of crosslinking may be used to gain precise control of extent of the physical extent of the hydrogel barrier as well as the degree of crosslinking, as described in the following section and in the Examples.

Inhibitors may be included within the flowable polymer solution to ensure precise control of photopatterning and to prevent extraneous or undesired polymerization. One useful inhibitor is hydroquinone monomethyl ether (MEHQ), but other suitable inhibitors may be used, such as 4-hydroxy TEMPO, or sodium ascorbate. The inhibitor may be present in the flowable polymer solution at a concentration of about 1 millimolar, about 2 millimolar, about 5 millimolar, about 10 millimolar, about 15 millimolar, about 20 millimolar, about 25 millimolar, about 30 millimolar, about 35 millimolar, about 40 millimolar or more, as needed to provide the photopatterning control desired.

Tuneable permeability. One aspect of performing assays using a hydrogel in a situ-generated isolation structure may include controlling the access of a molecule to an area of interest (e.g., completely exclude or control the rate of diffusion across the hydrogel). In some embodiments, the porosity of the in situ-generated structure restricts but does not block passage of an assay reagent. The in situ-generated structure may have selective permeability permitting one, some, or all of the one or more reagents to pass through the in situ-generated structure. In some embodiments, the in situ-generated structure may have a selective permeability that decreases the permeability of a reagent when it is bound with one or more reagents in the assay. As used herein, “porosity” describes an average pore size of an in situ-generated structure, and “permeability” describes how easily a molecule of interest passes through the in situ-generated structure. A molecule of interest can pass through the in situ-generated structure easier or faster if the in situ-generated structure has a high permeability to it. In contrast, the molecule of interest can pass through the in situ-generated structure more slowly if the permeability is lower. The permeability of the in situ-generated structure can be tuned to affect the diffusion of a molecule passing through.

Generally, the in situ-generated structure can be formed by introducing a prepolymer composition (e.g., a prepolymer solution) into the flow region and allowing the components of the prepolymer composition flow or diffuse to a selected area where the in situ-generated structure will be formed. The prepolymer composition can comprise a polymer molecule configured to form the in situ-generated structure, a crosslinker, an initiator, and an inhibitor as described herein. Selection of the chemical nature of the polymer (for example, molecular weight range, number of cross linkable moieties per polymer unit (linear, 2 arm, 4 arm, 8 arm, star or comb polymer), mixtures of polymers), the amount of the component (the polymer molecule, the crosslinker, the initiator, and/or the inhibitor), and mode of polymerization are variables that may be modified to tune the hydrogel barrier formed.

Generally, the initiator is a photoinitiator. Photopatterning provides precise control of the geometry of the polymerization as well as the extent of polymerization, and changes in exposure time and power of the illumination also can provide more control to arrive at a desired type of porosity and degree of robustness of the polymerized feature.

Mixtures of two different flowable polymers having similar molecular weight were found to be advantageous in providing hydrogel barriers with differential permeability. Using polymers having similar molecular weights confers similar rates of diffusion, which simplifies delivery to the region within the chamber. Since chambers that are sequestration pens are unswept regions of the microfluidic device, introduction of the polymer into the sequestration pen occurs substantially only by diffusion.

In many variations, polymer selection may depend upon the biocompatibility of the polymer species, and may be related to the specific application to which a hydrogel in situ-generated structure may be used. In some variations, the hydrogel may be a polyethylene glycol polymer or a modified polyethylene glycol polymer.

A wide range of molecular weights of the flowable polymer maybe suitable, depending upon the structure of the polymer. In some embodiments, the flowable polymer may have a molecular weight of about 500 Da to about 20 kDa, or about 500 Da, about 1 kDa, about 3 kDa, about 5 kDa, about 10 kDa, about 12 kDa, about 15 kDa, about 20 kDa or any value therebetween. A useful star type polymer may have Mw (weight average molecular weight) in a range from about 500 Da to about 20 kDa (e.g., four arm polymer), or up to about 5 kDa for each arm, or any value therebetween. In some embodiments, a polymer having a higher molecular weight range, may be used at lower concentrations in the flowable polymer, and still provide an in situ-generated barrier or isolation structure that may be used in the methods described herein.

In some practical embodiments, the concentration of the component (the polymer molecule, the crosslinker, the initiator, and/or the inhibitor) of the hydrogel composition can be varied to adjust the porosity of the hydrogel formed thereby tuning the permeability thereof. The concentration of a component can be tuned to be about −95%, −90%, −80%, −70%, −60%, −50%, −40%, −30%, −20%, −10%, −5%, +5%, +10%, +20%, +30%, +40%, +50%, +60%, +70%, +80%, +90%, +100%, +150%, +175%, +200%, +225%, +250%, +275%, +300%, +350%, +400%, or higher, or any value therebetween of a standard concentration. A standard concentration of a certain component can be determined based on a need of an assay and is not limited. The present disclosure teaches, once a standard concentration is determined, the permeability of the hydrogel can be tuned according to the disclosure herein.

In some embodiments, increasing the amount of a component of the prepolymer composition decreases the pore size of the hydrogel formed thereby slowing down diffusion of an assay reagent. For example, increasing the amount of a polymer molecule can result in a denser solidified polymer network that slows down the diffusion. In some embodiments, increasing the amount of a component increases the pore size of the hydrogel formed thereby accelerating diffusion of an assay reagent. For example, increasing the amount of an inhibitor can result in a less dense solidified polymer network whereby an assay reagent can diffuse faster (e.g., diffusion is slowed to a lesser degree by the barrier). In yet some other embodiments, the porosity of a hydrogel formed at a standard amount does not restrict passage of a molecule, therefore adjusting the amount of the components to get a less dense solidified polymer network would not affect the diffusion of the molecule.

In some embodiments, an in situ-generated structure comprises a porosity that can restrict passage of a molecule no smaller than a critical size (e.g. a cut-off value or threshold of the size of the molecule), and the porosity of the in situ-generated structure can be adjusted as described herein so that the diffusion rate of the molecule passing through the in situ-generated structure can be tuned. The critical size can be 10 kDa, 20 kDa, 30 kDa, 40 kDa, 50 kDa, 60 kDa, 70 kDa, 80 kDa, 90 kDa, 100 kDa, 150 kDa, 200 kDa, 250 kDa, 300 kDa, 350 kDa, 400 kDa, 450 kDa, 500 kDa, 550 kDa, 600 kDa, 650 kDa, 700 kDa, 750 kDa, 800 kDa, 850 kDa, 900 kDa, 950 kDa, 1000 kDa, or higher, or any value therebetween.

In a specific example, permeability of two reagents passing through an in situ-generated structure (a hydrogel barrier) was tested in a microfluidic device as described herein. In this example, a permeability test mixture comprising a mixture of AF647 labeled ovalbumin (AF647-OVA; Invitrogen™, #O34784) and AF488Plus labeled IgG (AF488P-IgG; Invitrogen™, #A32723) was prepared. The ovalbumin and the IgG in those reagents had a molecular weight of 45 kDa and 150 kDa respectively, and their final concentration in the permeability test mixture was 0.02 mg/mL and 0.05 mg/mL respectively.

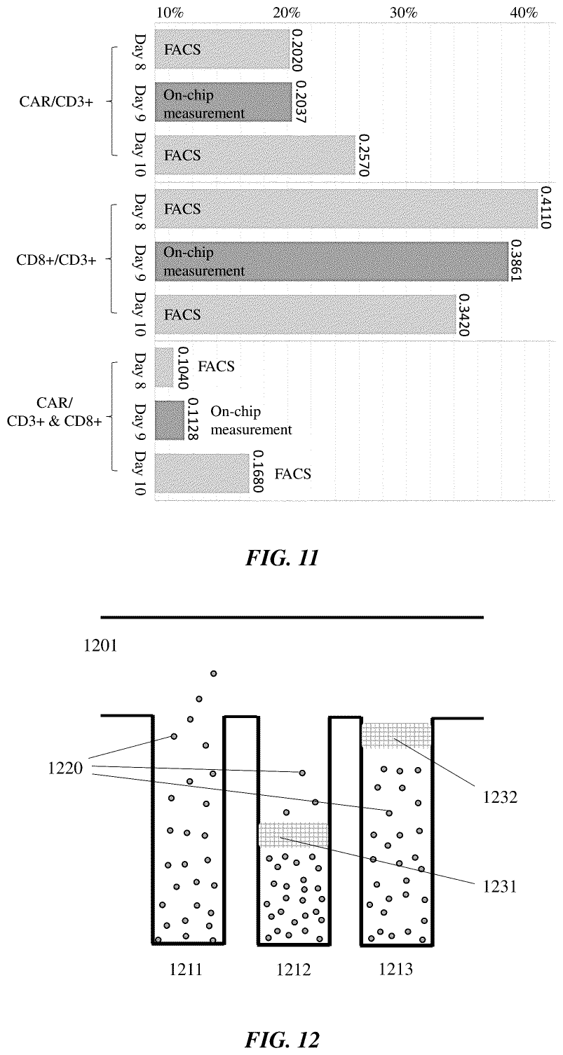

The microfluidic device was first calibrated and flushed with PBS. Then, the permeability test mixture was introduced while there was no hydrogel structure formed within the device. The perfusion of the permeability test mixture was continued for one hour to let the reagents diffuse into the chambers of the microfluidic device. Time-lapsed brightfield images and fluorescent images were taken. See FIG. 13A, showing Time 0 when the permeability test mixture was just introduced into the channel 1301. The channel 1301 had a bright fluorescent image due to the presence of the fluorescent label of the AF647-OVA, while the chambers 1302 were still dark because the permeability test mixture had not yet diffused into the chambers 1302. The images of AF488P-IgG were also taken but not shown in this figure. After 60 minutes, the permeability test mixture had diffused and reached equilibration across the channel 1301 and the chambers 1302 so that the channel 1301 and the chambers 1302 were about equally bright in the image (FIG. 13B).

Then, the microfluidic device was flushed with PBS to remove the permeability test mixture remaining therewithin. A prepolymer composition comprising 8A10K PEG norbornene (the prepolymer A in FIG. 13), 8A20K PEG norbornene (the prepolymer B in FIG. 13), a crosslinker (dithiothreitol), an initiator (LAP), and an inhibitor (sodium ascorbate) at a standard concentration for in situ-generation of a hydrogel barrier was introduced into the microfluidic device. Solidification of the prepolymer composition was activated by illuminating UV light at a selected area within each chamber 1302 close to the opening 1303. The in situ-generated hydrogel barrier 1310 formed and sealed the opening 1303 as shown in FIG. 13C. Then, the permeability test mixture was introduced again into the channel 1301 and allowed to diffuse into the chambers 1302 for 60 minutes. With the in situ-generated hydrogel barrier 1310 in place, the permeability test mixture must pass through it to reach the chambers 1302.

FIG. 13D shows an image at Time 0 wherein the permeability test mixture was just introduced. The channel 1301 was had a bright fluorescent image due to the presence of the fluorescent label of the AF647-OVA, while the chambers 1302 were still dark because the permeability test mixture had not yet diffused into the chambers 1302. Compared with FIG. 13A, the chambers 1302 in FIG. 13D were darker showing that the in situ-generated hydrogel barrier 1310 impeded the diffusion of the permeability mixture. In contrast, the permeability test mixture can quickly diffuse into the chambers 1302 in FIG. 13A even just upon introducing the mixture into the flow region.

After diffusion for 60 minutes, most permeability test mixture still remained in the channel 1301. As the chambers 1302 were brighter compared with that in FIG. 13D, indicating that the in situ-generated hydrogel barrier 1310 did not block diffusion but slowed down the diffusion of the permeability mixture, which can also be seen clearly from the comparison between FIG. 13E and FIG. 13B.

Alternatively, the final concentration of each component of the hydrogel solution was adjusted to tune the permeability of the permeability mixture. The intensities of the fluorescent signals of the permeability test mixture (AF647-OVA and AF488P-IgG respectively) detected from regions of interest in channel and in chambers were detected to calculate channel-to-chamber ratios. At time 0 when there was no permeability test mixture existing within the microfluidic device, the ratio should be close to 0. At full equilibration (the substantial difference in concentration of the permeability test mixture across the channel and the chamber), the ratio should be close to 1. The ratios were used in the following exponential equation to calculate a diffusion coefficient representing diffusion behavior in various conditions. The I_{chamber}/I_{channel} represents the channel-to-chamber ratio as described above; t is time; D is the diffusion coefficient.

$${I}_{c h a m b e r} / {I}_{c h a nnel} = 1 - e \hat{} ( - D * t )$$

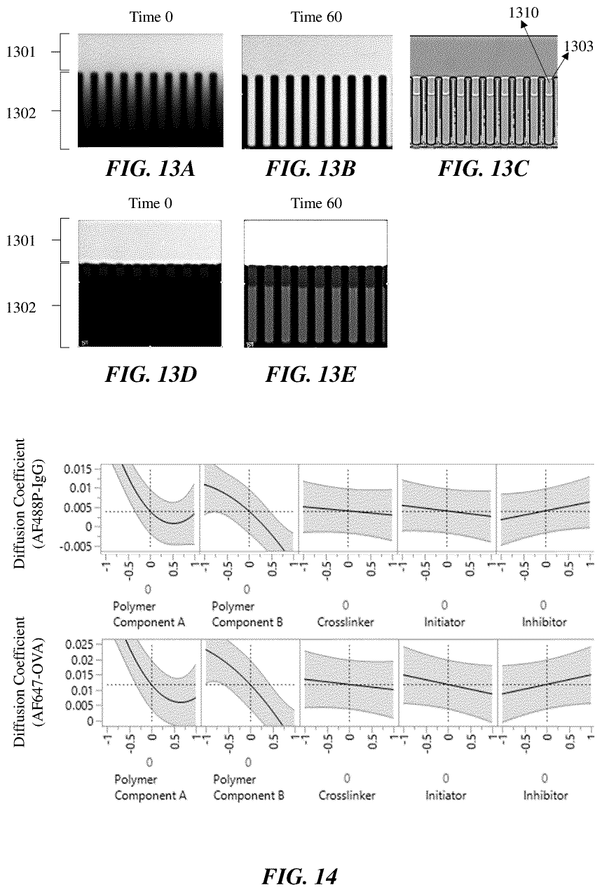

The concentration of each component (prepolymer component A, prepolymer component B, crosslinker, initiator, and inhibitor) of the hydrogel solution varied independently from −50% to 200% of the standard concentration. FIG. 14 shows that the diffusion coefficient varied along with variation of the concentration of each component. A strong relationship between hydrogel permeability and polymer concentration was observed. In both cases, as the concentration of polymer went up, the permeability of the hydrogel went down. In contrast, the concentrations of the crosslinker, the initiator, and the inhibitor only had a modest impact on the permeability. The data verifies that the permeability of hydrogel to a molecule of interest can be tuned by adjusting the concentration of the component of the hydrogel solution resulting in changes of the porosity of the hydrogel

Since the crosslinker is designed to be incorporated into the formed hydrogel structure, it is surprising that the concentration of the crosslinker is not the primary factor in determining the porosity of a hydrogel. Hydrogels of different porosities can be formed from prepolymer compositions comprising a same or similar density of crosslinkers. This is particularly beneficial for forming reversible hydrogel structures that can be dissolved by cleaving the crosslinkers in the hydrogel structures. For example, a first reversible hydrogel of high permeability and a second reversible hydrogel of low permeability may comprise a same cleavable crosslinker moiety. Even though the first reversible hydrogel and the second reversible hydrogel have different permeabilities, they can be dissolved with similar efficiencies using the same dissolution reagent.

In some embodiments where a microfluidic device comprising a microfluidic circuit material defining a flow region, a first chamber and a second chamber is used, the first chamber and the second chamber can have a first permeable hydrogel barrier and a second permeable hydrogel barrier formed therewithin respectively. The first permeable hydrogel barrier and the second permeable hydrogel barrier can have different physical properties, for example, different porosities or different permeabilities to a reagent. The first permeable hydrogel barrier and the second permeable hydrogel barrier can be both reversible hydrogel barrier, non-reversible hydrogel barrier, or one of them is a reversible hydrogel barrier and the other is a non-reversible hydrogel barrier.

In some embodiments, the first permeable hydrogel barrier has a different permeability from a permeability of the second permeable hydrogel barrier. In some embodiments, the first permeable hydrogel barrier has a permeability limit that is higher than a permeability limit of the second permeable hydrogel barrier. A reagent configured to diffuse through the first permeable hydrogel barrier may be excluded from diffusing through the second permeable hydrogel barrier, when that reagent has a size greater than the permeability limit of the second permeable hydrogel barrier. In some other embodiments, a reagent configured to diffuse through the first permeable hydrogel barrier and the second permeable hydrogel barrier might diffuse at a different rate passing through the first permeable hydrogel barrier and the second permeable hydrogel barrier. For example, a first rate of the reagent to pass through the first permeable hydrogel barrier might be higher than a second rate of the reagent to pass through the second permeable hydrogel barrier.

Forming the first permeable hydrogel barrier may include forming the first permeable hydrogel barrier within one or more chambers of the plurality of sequestration pens. Forming the first permeable hydrogel barrier may further include forming the first permeable hydrogel barrier distal to or at the opening of the one or more chambers, thereby producing one or more reversibly capped chambers.

A process for forming the first permeable hydrogel barrier and the second permeable hydrogel barrier can comprise: introducing a first prepolymer composition into the flow region of the microfluidic device, where the first prepolymer composition has a first selected set of characteristics defining physical properties of a first permeable hydrogel barrier formed therefrom; diffusing the first prepolymer composition from the flow region into the plurality of chambers; activating crosslinking of the first prepolymer composition in a selected area of the microfluidic device, thereby forming a first permeable hydrogel barrier within the flow region or one or more of the plurality of chambers of the microfluidic device.

The process can further comprise introducing a second prepolymer composition into the flow region of the microfluidic device, where the second prepolymer composition has a second selected set of characteristics defining physical properties of a second permeable hydrogel barrier formed therefrom; diffusing the second prepolymer composition from the flow region into the chambers; and activating crosslinking of the second prepolymer composition in a selected area of the microfluidic device, thereby forming a second permeable hydrogel barrier within the flow region or a sub-set of the plurality of chambers of the microfluidic device.

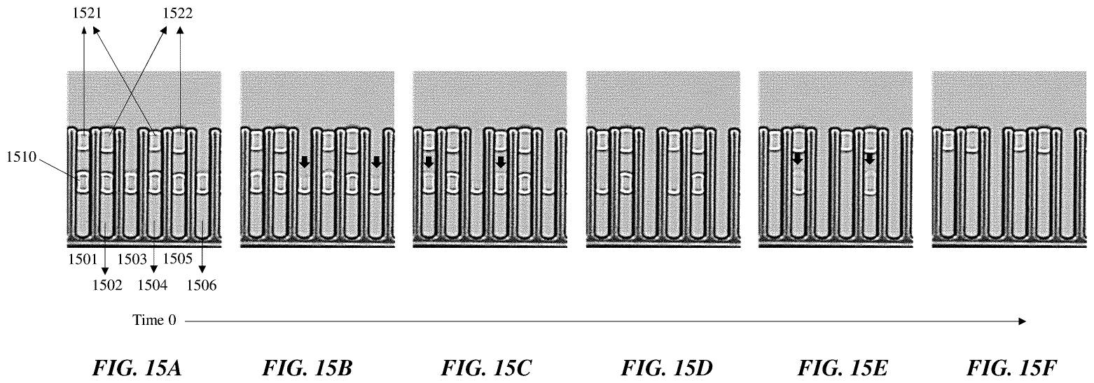

In a specific example, a reversible hydrogel barrier 1510 was formed at the mid-pen location (mid-area) of each chamber, a first non-reversible hydrogel barrier 1521 was formed in chamber 1501 and chamber 1504 respectively, a second non-reversible hydrogel barrier 1522 was formed in chamber 1502 and chamber 1505 respectively (FIG. 15A).

The reversible hydrogel barrier 1510 was formed by using a prepolymer composition comprising 8A10K PEG norbornene, 8A20K PEG norbornene, a crosslinker (a peptide: GCRDLPRTGGDRCG), an initiator (LAP), and an inhibitor (sodium ascorbate).

Both the first non-reversible hydrogel barrier 1521 and second non-reversible hydrogel barrier 1522 were formed by using a first prepolymer composition and a second prepolymer composition respectively. The first prepolymer composition and the second prepolymer composition comprises same components: 8A10K PEG norbornene, 8A20K PEG norbornene, a crosslinker (sodium 2,3-dimercaptopropanesulfonate), an initiator (LAP), and an inhibitor (sodium ascorbate), but the second prepolymer composition comprises the 8A10K PEG norbornene and the 8A20K PEG norbornene at a concentration 2-fold the concentration of that in the first prepolymer composition.

A dissolution reagent (TrypLE™; 24 kDa) was introduced into the channel of the microfluidic device and allowed to diffuse into those chambers. The dissociation of the reversible hydrogels barrier 1510 was recorded. Six images were extracted from the recording including an image at Time 0 (FIG. 15A) and images of other five different timepoints (FIGS. 15B to 15F).

It was observed that the reversible hydrogel barrier 1510 within chamber 1503 and chamber 1506 started to be dissolved (almost immediately after the dissolution reagent was introduced) while the reversible hydrogels barrier 1510 within other chambers remained substantially intact (See FIG. 15B, the arrows indicate the dissociation of the hydrogels). Along time, the reversible hydrogels barrier 1510 in chamber 1501 and chamber 1504 started to be dissolved (at about 4 min after the dissolution reagent was introduced. See FIG. 15C, the arrows indicate the dissociation of the hydrogels). The reversible hydrogels barrier 1510 in chamber 1503 and chamber 1506 were about half gone at this moment while no substantial change was observed in the reversible hydrogel barriers 1510 in chamber 1502 and chamber 1505. Later in FIG. 15D, the reversible hydrogels barrier 1510 in chamber 1503 and chamber 1506 were dissolved completely (at about 12 min after the dissolution reagent was introduced), and so were most of the reversible hydrogel barriers 1510 in chamber 1501 and chamber 1504. Then in FIG. 15E, the reversible hydrogel barriers 1510 in chamber 1503 and chamber 1506 started to be dissolved (at about 22 min after the dissolution reagent was introduced) and eventually dissolved completely as shown in FIG. 15F (at about 60 min after the dissolution reagent was introduced).

According to the images, the second non-reversible hydrogel barrier 1522 having higher concentration of the polymer molecule had a porosity impeding passage of the dissolution reagent greater than the first non-reversible hydrogel barrier 1521. In other words, the permeability of the second non-reversible hydrogel barrier 1522 to the dissolution reagent was lower than that of the first non-reversible hydrogel barrier 1521 whereby the dissolution reagent diffused more slowly in chamber 1502 and chamber 1505. The result verifies that, by adjusting the concentration of the polymer molecule used for forming the in situ-generated hydrogel barrier, the porosity of the hydrogel barrier formed can be tuned resulting different in permeability to a molecule of interest.

Reversing/removing/minimizing the in situ-generated structure. A number of mechanisms may be used to remove or reduce the in situ-generated structure when there is no further purpose for it. In some other embodiments, the in situ-generated structure is removed or reduced for performing an assay.

Mechanical force. Increasing flow can be used if at least a portion of the hydrogel barrier is located within a flow region as opposed to an isolation region of a pen. For example, the at least one isolation structure may be located within an isolation region of a sequestration pen, and after the assay is complete, the sequestration pen or the isolation region therein may be modified to bring flow through the isolation region. In other variations, such as is described herein, laser initiated bubbles may provide forces that can deform or disrupt the hydrogel barrier, permitting export of the cells.

Hydrolytic susceptibility. Porogens, including polymers which are incapable of being chemically linked to the photoinitiated polymer(s), may be included when forming the hydrogel barrier. The degree/size of openings within the formed hydrogel can customize the hydrolysis rate via accessibility within the hydrogel barrier). In other embodiments, the pores formed may be employed to permit secreted materials or chemical reagents to pass through the hydrogel barrier but prevent a cell from moving into, out of, and/or through the isolation structure. In other embodiments, degradability of these polymers may be increased by introducing degradable segments such as polyester, acetal, fumarate, poly(propylene fumarate) or polyhydroxyacids into polymers (e.g., PEG polymers).

Reducing agents: PEG may be formed with disulfide linkages at intervals along the macromere, which may be random or predetermined. The disulfide bonds may be broken by Dithiothreitol (DTT), mercaptoethanol, or TCEP.

Thermal, poly N-isopropylacrylamide (PNIPAm) or other suitable LCST polymers may be used to introduce hydrogel barriers upon heating. They may be removed by decreasing the temperature of the formed polymer hydrogel barrier. The polymers may include ELPs or other motifs that also permit removal by other mechanisms such as hydrolysis or proteolysis.

Proteolytic susceptibility: Hydrogels may have any sort of peptide sequence engineered in, such that selective proteolysis upon a selected motif by a selected protease can remove/reverse/or minimize a hydrogel isolation structure. Some classes of modified PEG include PEG having elastin like peptide (ELP) motifs and/or having peptide motifs for susceptibility to a variety of proteases (enzyme sensitive peptide ESP). A large number of these motifs are known. One useful motif is RGD which may be constrained to be cyclic.

In some embodiments, when the composition has a single type of crosslinker molecule comprising a vic-diol moiety or a peptide moiety, the method may further include removing the hydrogel barrier at a selected time by introducing a reversing reagent into the flow region and diffusing the reversing reagent into the sequestration pens, thereby removing the hydrogel barrier and providing one or more uncapped sequestration pens. The reversing reagent is a periodate reagent configured to cleave the vic-diol moiety or an enzyme configured to cleave the peptidyl moiety. In some embodiments, the enzyme configured to cleave the peptidyl moiety is a trypsin enzyme or analog thereof. The analog of the trypsin enzyme may be a TrypLE™ enzyme. The method may further include unpenning one or more biological micro-objects from at least one uncapped chamber.

Osmotic susceptibility. Calcium concentration/other osmotic strategies can be employed to degrade and remove a hydrogel barrier. As above, changes of media flowed through the channel or flow region may dimensionally swell or de-swell hydrogel barriers.

Photocleavage. As described above, if a polymer of the solidified polymer network includes a photocleavable moiety, directing illumination of an exciting wavelength to the solidified polymer network will cause cleavage within sections of the solidified polymer network. This cleavage may provide complete or partial disruption of the solidified polymer network, thereby removing or reducing the hydrogel barrier.

Chemical susceptibility. As described in the sections above directed to thiol-ene polymers, specific moieties of the hydrogel polymer may be susceptible to chemical cleavage, such as reducing agents or periodate cleavage.

In some applications, the hydrogel barrier may not be removed but may simply be swelled or de-swelled using light or media\solvent changes. Some types of hydrogels may incorporate moieties that respond reversibly to light (for example, change regiochemistry about a rigid bond; form reversible crosslinks within the polymer, or form/break ion pairs).

### Applications of the Method of the Present Disclosure

Staining assay. In one embodiment, the method of the present disclosure can be used to stain a biological micro-object within a microfluidic device. For example, the staining can be conducted to observe the viability of a biological micro-object. In such an example, the assay reagent can be a mitochondrial potential reagent. Examples of reagents for testing viability include but not limited to Annexin V, propidium iodide, an AOPI dye, or a combination thereof. In some embodiments, an AOPI dye can be used in these methods since the micro-objects may be cells. These dyes contain a mixture of acridine orange (AO) and propidium iodide (PI). Acridine orange (AO) and propidium iodide (PI) are nuclear staining (or nucleic acid binding) dyes. AO is permeable to both live and dead cells and stains all nucleated cells to generate green fluorescence. PI enters dead cells with compromised membranes and stains all dead nucleated cells to generate red fluorescence. Cells stained with both AO and PI fluoresce red due to Forster resonance energy transfer (FRET), so all live nucleated cells fluoresce green, and all dead nucleated cells fluoresce red.

In other embodiments, the staining can be conducted to observe a surface marker on a biological micro-object associated with proliferation, activation, metabolic activity, phenotype, exhaustion, and/or lineage. In certain embodiments, the assay reagent comprises a binding agent specific to the micro-object and a detectable label. The binding agent can be an antibody that is able to recognize CD3, CD4, CD8, CD14, CD19, or CD25. In some embodiments, the detectable label can be a colorimetric label, a fluorescent label, or a luminescent label.

In some embodiments, surface staining may be performed for markers such as: CD4, CD8, CD137, CD107a, and/or CD69, each indicator of classification status of a T cell. In other embodiments, surface staining may be performed for the presence of CD28, CD127, CCR7, and/or CD107. Proliferation (fold-expansion or cell divisions) may be examined for either a T cell or for a target cell by CFSE staining or Live-Dead stain. Whether the T cell has a memory phenotype may be interrogated by use of surface or intracellular stain for one or more of CD45RA, CD45RO, CCR7. Further, metabolic state may be interrogated using Mitoview or 2-NBDG. The status of the T cell may be further probed for exhaustion by use of surface stains for markers such as CD57, KLRG1, TIM-3, LAG-3, and/or PD-1. Additionally, the lineage of the T cell may be probed, such whether it is TH1/TH2, CD4 and the like by looking at the cytokine production, release and/or capture of IFNgamma, IL-4, and/or IL13 release or by performing transcriptional profiling of IFNgamma, IL-4, and/or IL-13 expression.

Such a staining assay can be helpful for the manufacturing of a cell therapeutic reagent comprising engineered T cells, chimeric antigen receptor T cells, tumor infiltrating cells, natural killer cells, or combinations thereof. Those cell therapeutic reagents are usually manufactured by isolating a certain type of cells from a patient and culturing and manipulating the isolated cells ex vivo to equip the isolated cells with a desired feature. The manipulated cells will be introduced directly back to the patient to cure the disease, so that the culture environment (e.g., a bioreactor) needs to be carefully controlled to avoid contamination. Nevertheless, the manipulated cells still need to be verified if they successfully present the desired feature before being introduced back to the patient. Therefore, a subset of the manipulated cells will be removed from the culture environment for testing. By using the method of the present disclosure, the subset of the manipulated cells can be verified effectively (for instance, stained) for the viability and/or the surface markers thereof without contacting the whole population of the manipulated cells with the assay reagents.

Cytotoxicity assay. In one embodiment, the method of present invention can be used to measure the ability of a potential cytotoxic reagent to affect the viability of a biological micro-object. For example, the biological micro-object can be a cancer cell, and the assay reagent can be an anticancer agent. The anticancer agent can be but not limited to a chemical, a peptide, a protein, or a combination thereof. In some embodiments, the assay reagent is a cytokine, including but not limited to TNF alpha, TGF beta, INF gamma, IL1 beta, IL2, IL4, IL6, IL10, IL12, IL13, IL17A, IL22, GM-CSF, or any combination thereof. In another example, the biological micro-object can be an insect cell or a plant cell, and the assay reagent can be a pesticidal agent including an insecticide, insect repellent, antifeedant, molluscicide, fungicide, phytotoxin, or a combination thereof.

The interaction between the assay reagent and the biological micro-object can be observed by observing a morphological change of the biological micro-object. The morphological change can be regarded to one or more physical characteristics (e.g., size and/or shape) associated with cell viability, which can be directly observed using a microscopy. In some embodiments, the morphological change can be identified using a reporter reagent. The reporter reagent can be a dye as described above, or Lactate dehydrogenase (LDH) assay, trypan blue exclusion and other live/dead staining reagents. In some embodiments, the reporter reagent can comprise a cell cytoplasm-localized fluorescent probe (CLFP) that only stain cell cytoplasm of living cells.

In some embodiments, the interaction between the assay reagent and the biological micro-object can be observed by using a reporter reagent that detects an activity of the biological micro-object associated with viability, for example, a 3-(4,5-dimethylthiazolyl-2)-2,5-diphenyl-tetrazolium bromide (MTT) assay. In certain embodiments, the reporter reagent can be a reporter of apoptosis. For example, Caspase-8 is produced and active between early and late stages of apoptosis. The Vybrant™ FAM Caspase-8 Assay Kit is based on a fluorescent inhibitor of caspases (FLICA™) methodology, essentially an affinity label. The reagent associates a fluoromethyl ketone (FMK) moiety, which can react covalently with a cysteine, with a caspase-specific amino acid sequence. For caspase-8, this recognition sequence is leucine-glutamic acid-threonine-aspartic acid (LETD). A fluorescein group is attached as a reporter. The FLICA reagent is thought to interact with the enzymatic reactive center of an activated caspase via the recognition sequence, and then to attach covalently through the FMK moiety. Fluorescence intensity in the FITC channel is directly proportional to the levels of caspase-8 in the cell and is a direct reporter.

Chemotaxis assay. The method of the present disclosure can also be used to observe how a biological micro-object reacts to a molecule potentially chemotactic to the biological micro-object. Without wishing being bound by theory, by using a chamber of the microfluidic device as a reservoir of a reagent and allowing the reagent to diffuse out of the chamber while the biological micro-object is present in the flow region, the diffusion creates a concentration gradient of the reagent. If the reagent is chemotactic to the biological micro-object, the biological micro-object would be attracted and migrate toward the chamber where the reagent is present at a higher concentration compared with the flow region. The migration can be viewed as an outcome of the interaction between the micro-object and the assay reagent and can be observed by observing the relative position of the micro-object in two different time points or by observing a morphological change of the micro-object (e.g., elongated cell shape).

An in situ-generated structure can be particularly useful in this application. For example, an in situ-generated cap can be formed to seal the chamber as described above that the porosity of the in situ-generated cap impedes the diffusion of the assay reagent. The in situ-generated cap can slow down the diffusion rate of the assay reagent resulting in a steeper gradient of the assay reagent concentration across the chamber and the flow region. The in situ-generated cap can also prevent the assay reagent from escaping from the chamber too quickly to show the chemotactic effect to the micro-object.

The chemotactic reagent can be a chemokine or growth factor, including but not limited to CXCL1, CXCL5, CXCL6, CXCL8, CXCL12, CX3CL1, CCL2, CCL3, CCL5, CCL19, CCL21, CCL22, CX3CL1, EGDF, TGF alpha, TGF beta, betacellulin, HBEGF, amphiregulin, heregulin, FGF, PDGF, IGF, CSF1, VEGFA, or VEGFC, that might be chemotactic to a cancer cell so that the method of present invention can be used to observe the migration of cancer cells. In some embodiments, a second assay reagent that is a potential anti-metastasis agent can be introduced into the flow region and the migration of the cancer cells can be observed to determine the efficacy of the potential anti-metastasis agent.

Microfluidic device/system feature cross-applicability. It should be appreciated that various features of microfluidic devices, systems, and motive technologies described herein may be combinable or interchangeable. For example, features described herein with reference to the microfluidic device 100, 175, 200, 300, 320, 400, 450, 520 and system attributes as described in FIGS. 1A-5B may be combinable or interchangeable.

Microfluidic devices. FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120, forming a three-layer structure.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include sub-classes including sequestration pens), traps, and the like. Circuit elements can also include barriers, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material. However, the microfluidic circuit structure need not include a frame 114. For example, the microfluidic circuit structure can consist of (or consist essentially of) the microfluidic circuit material 116.

The microfluidic circuit material 116 can be patterned with cavities or the like to define the circuit elements and interconnections of the microfluidic circuit 120, such as chambers, pens and microfluidic channels. The microfluidic circuit material 116 can comprise a flexible material, such as a flexible polymer (e.g., rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g., photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and/or fluidically connected thereto. A chamber can have one or more openings fluidically connecting the chamber with one or more flow regions. In some embodiments, a flow region comprises or corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings. In some embodiments of sequestration pens, a sequestration pen may have only a single opening in fluidic communication with the flow path 106. In some other embodiments, a sequestration pen may have more than one opening in fluidic communication with the flow path 106, e.g., n number of openings, but with n−1 openings that are valved, such that all but one opening is closable. When all the valved openings are closed, the sequestration pen limits exchange of materials from the flow region into the sequestration pen to occur only by diffusion. In some embodiments, the sequestration pens comprise various features and structures (e.g., isolation regions) that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure. Regardless of the various possible integrations, the microfluidic device can retain a three-layer structure that includes a base layer and a cover layer that sandwich a middle layer in which the microfluidic circuit 120 is located.

In some embodiments, the cover 110 can comprise a rigid material. The rigid material may be glass, a plastic, or a material with similar properties to either of the foregoing. In some embodiments, the cover 110 can comprise a deformable material. The deformable material can be a polymer, such as PDMS or variations thereof known in the art. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. Microfluidic devices having covers that include both rigid and deformable materials have been described, for example, in U.S. Pat. No. 10,058,865 (Breinlinger et al.), the contents of which are incorporated herein by reference. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the sequestration pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the sequestration pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single flow path 106 containing a single channel 122 is shown. However, other embodiments may contain multiple channels 122 within a single flow path 106, as shown in FIG. 1B. The microfluidic circuit 120 further comprises an inlet valve or port 107 in fluid communication with the flow path 106, whereby fluidic medium 180 can access the flow path 106 (and channel 122). In some instances, the flow path 106 comprises a substantially straight path. In other instances, the flow path 106 is arranged in a non-linear or winding manner, such as a zigzag pattern, whereby the flow path 106 travels across the microfluidic device 100 two or more times, e.g., in alternating directions. The flow in the flow path 106 may proceed from inlet to outlet or may be reversed and proceed from outlet to inlet.

One example of a multi-channel device, microfluidic device 175, is shown in FIG. 1B, which may be like microfluidic device 100 in other respects. Microfluidic device 175 and its constituent circuit elements (e.g., channels 122 and sequestration pens 128) may have any of the dimensions discussed herein. The microfluidic circuit illustrated in FIG. 1B has two inlet/outlet ports 107 and a flow path 106 containing four distinct channels 122. The number of channels into which the microfluidic circuit is sub-divided may be chosen to reduce fluidic resistance. For example, the microfluidic circuit may include 1, 2, 3, 4, 5, 6, 7, 8, 9, 10, or more channels to provide a selected range of fluidic resistance. Microfluidic device 175 further comprises a plurality of sequestration pens opening off of each channel 122, where each of the sequestration pens is similar to sequestration pen 128 of FIG. 1A, and may have any of the dimensions or functions of any sequestration pen as described herein. However, the sequestration pens of microfluidic device 175 can have different shapes, such as any of the shapes of sequestration pens 124, 126, or 130 of FIG. 1A or as described anywhere else herein. Moreover, microfluidic device 175 can include sequestration pens having a mixture of different shapes. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object. In some instances, the trap 132 comprises a side passage 134 that is smaller than the target micro-object in order to facilitate flow through the trap 132.

Sequestration pens. The microfluidic devices described herein may include one or more sequestration pens, where each sequestration pen is suitable for holding one or more micro-objects (e.g., biological cells, or groups of cells that are associated together). The sequestration pens may be disposed within and open to a flow region, which in some embodiments is a microfluidic channel. Each of the sequestration pens can have one or more openings for fluidic communication to one or more microfluidic channels. In some embodiments, a sequestration pen may have only one opening to a microfluidic channel.

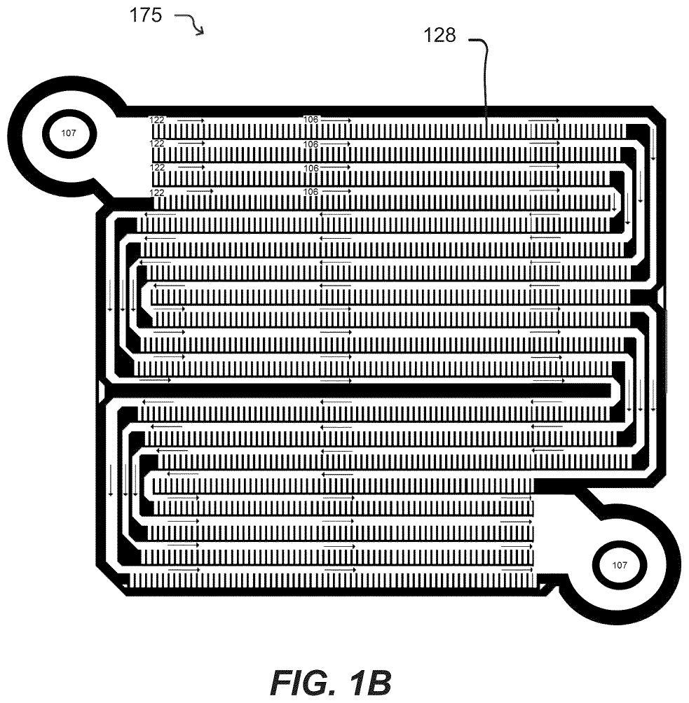

FIGS. 2A-2C show sequestration pens 224, 226, and 228 of a microfluidic device 200, which may be like sequestration pen 128 of FIG. 1A. Each sequestration pen 224, 226, and 228 can comprise an isolation region 240 and a connection region 236 fluidically connecting the isolation region 240 to a flow region, which may, in some embodiments include a microfluidic channel, such as channel 122. The connection region 236 can comprise a proximal opening 234 to the flow region (e.g., microfluidic channel 122) and a distal opening 238 to the isolation region 240. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, streamlines from the flow in the microfluidic channel do not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which depicts a vertical cross-section of microfluidic device 200. FIG. 2B shows a horizontal cross-section of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be equal to or less than about 10%, 7%, 5%, 3%, 2%, 1%. 0.9%, 0.8%, 0.5%, 0.3% or 0.1% of the height of the walls of the sequestration pen. Alternatively, the variation of elevation in the upper surface of the substrate across both the microfluidic channel 122 (or flow region) and sequestration pens may be equal to or less than about 2%, 1%. 0.9%, 0.8%, 0.5%, 0.3%, 0.2%, or 0.1% of the height of the substrate. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. Sequestration pens like 224, 226, 228 have isolation regions wherein each isolation region has only one opening, which opens to the connection region of the sequestration pen. Fluidic media exchange in and out of the isolation region so configured can be limited to occurring substantially only by diffusion. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 (see FIG. 2C) of fluidic medium 180 in the microfluidic channel 122 can be selectively generated and stopped. For example, as shown, the ports 222 can be disposed at different locations (e.g., opposite ends) of the flow region (microfluidic channel 122), and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of the connection region 236 of sequestration pen 224 can cause a secondary flow 244 of the fluidic medium 180 into and out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ depends upon a number of factors, including the shape of the microfluidic channel 122, which may be defined by a width W_{con} of the connection region 236 at the proximal opening 234; a width W_{ch} of the microfluidic channel 122 at the proximal opening 234; a height H_{ch} of the channel 122 at the proximal opening 234; and the width of the distal opening 238 of the connection region 236. Of these factors, the width W_{con} of the connection region 236 at the proximal opening 234 and the height H_{ch} of the channel 122 at the proximal opening 234 tend to be the most significant. In addition, the penetration depth Dₚ can be influenced by the velocity of the fluidic medium 180 in the channel 122 and the viscosity of fluidic medium 180. However, these factors (i.e., velocity and viscosity) can vary widely without dramatic changes in penetration depth Dₚ. For example, for a microfluidic chip 200 having a width W_{con} of the connection region 236 at the proximal opening 234 of about 50 microns, a height H_{ch} of the channel 122 at the proximal opening 122 of about 40 microns, and a width W_{ch} of the microfluidic channel 122 at the proximal opening 122 of about 100 microns to about 150 microns, the penetration depth Dₚ of the secondary flow 244 ranges from less than 1.0 times W_{con} (i.e., less than 50 microns) at a flow rate of 0.1 microliters/sec to about 2.0 times W_{con} (i.e., about 100 microns) at a flow rate of 20 microliters/sec, which represents an increase in Dₚ of only about 2.5-fold over a 200-fold increase in the velocity of the fluidic medium 180.

In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-objects 246 out of the isolation region 240, which is an unswept region of the microfluidic circuit, resulting in the micro-objects 246 being retained within the isolation region 240. Accordingly, selection of microfluidic circuit element dimensions and further selection of the operating parameters (e.g., velocity of fluidic medium 180) can prevent contamination of the isolation region 240 of sequestration pen 224 by materials from the microfluidic channel 122 or another sequestration pen 226 or 228. It should be noted, however, that for many microfluidic chip configurations, there is no need to worry about Vₘₐₓ per se, because the chip will break from the pressure associated with flowing fluidic medium 180 at high velocity through the chip before Vₘₐₓ can be achieved.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94% 95%, 96%, 97%, 98%, or greater than about 99% of fluidic exchange.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g., a portion of the connection region adjacent to the proximal opening 234).

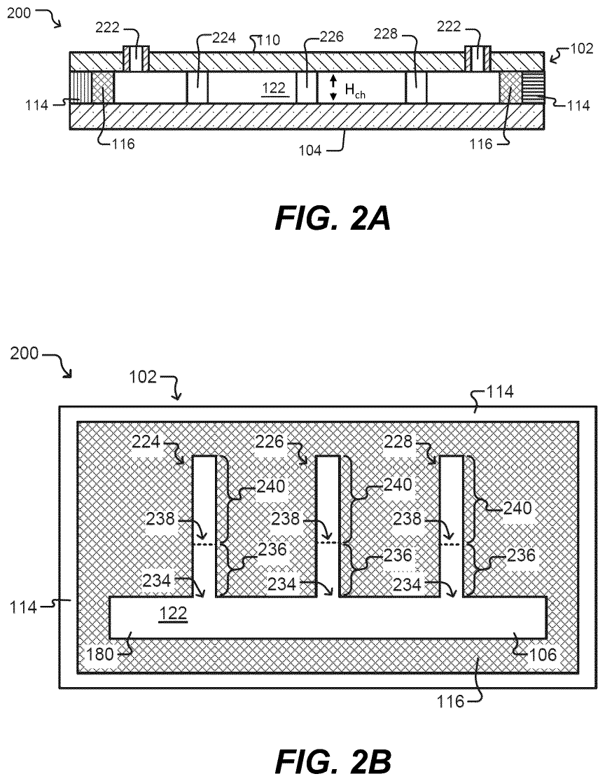

FIG. 3 depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 3 include a microfluidic channel 322, having a width W_{ch}, as described herein, and containing a flow 310 of first fluidic medium 302 and one or more sequestration pens 324 (only one illustrated in FIG. 3). The sequestration pens 324 each have a length Ls, a connection region 336, and an isolation region 340, where the isolation region 340 contains a second fluidic medium 304. The connection region 336 has a proximal opening 334, having a width W_{con1}, which opens to the microfluidic channel 322, and a distal opening 338, having a width W_{con2}, which opens to the isolation region 340. The width W_{con1} may or may not be the same as W_{con2}, as described herein. The walls of each sequestration pen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestration pen 324. In some embodiments, the length L_{con} of the connection region 336 is at least partially defined by length L_{wall} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestration pen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352.

In sequestration pens configured like those of FIGS. 2A-2C and 3, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n−1 openings can be valved. When the n−1 valved openings are closed, the isolation region has only one effective opening, and exchange of materials into/out of the isolation region occurs only by diffusion.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Microfluidic circuit element dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure.

For any of the microfluidic devices described herein, a microfluidic channel may have a uniform cross sectional height along its length that is a substantially uniform cross sectional height, and may be any cross sectional height as described herein. At any point along the microfluidic channel, the substantially uniform cross sectional height of the channel, the upper surface of which is defined by the inner surface of the cover and the lower surface of which is defined by the inner surface of the base, may be substantially the same as the cross sectional height at any other point along the channel, e.g., having a cross sectional height that is no more than about 10%, about 9%, about 8%, about 7%, about 6%, about 5%, about 4%, about 3%, about 2% or about 1% or less, different from the cross-sectional height of any other location within the channel.

Additionally, the chamber(s), e.g., sequestration pen(s), of the microfluidic devices described herein, may be disposed substantially in a coplanar orientation relative to the microfluidic channel into which the chamber(s) open. That is, the enclosed volume of the chamber(s) is formed by an upper surface that is defined by the inner surface of the cover, a lower surface defined by the inner surface of the base, and walls defined by the microfluidic circuit material. Therefore, the lower surface of the chamber(s) may be coplanar to the lower surface of the microfluidic channel, e.g., substantially coplanar. The upper surface of the chamber may be coplanar to the upper surface of the microfluidic channel, e.g., substantially coplanar. Accordingly, the chamber(s) may have a cross-sectional height, which may have any values as described herein, that is the same as the channel, e.g., substantially the same, and the chamber(s) and microfluidic channel(s) within the microfluidic device may have a substantially uniform cross sectional height throughout the flow region of the microfluidic device, and may be substantially coplanar throughout the microfluidic device.

Coplanarity of the lower surfaces of the chamber(s) and the microfluidic channel(s) can offer distinct advantage with repositioning micro-objects within the microfluidic device using DEP or magnetic force. Penning and unpenning of micro-objects, and in particular selective penning/selective unpenning, can be greatly facilitated when the lower surfaces of the chamber(s) and the microfluidic channel to which the chamber(s) open have a coplanar orientation.

The proximal opening of the connection region of a sequestration pen may have a width (e.g., W_{con} or W_{con1}) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or W_{con1}) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or Wₒₙ₁) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., L_{con}) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25. times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or W_{con1}) of the proximal opening. Thus, for example, the proximal opening of the connection region of a sequestration pen may have a width (e.g., W_{con} or W_{con1}) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or W_{con1}) from about 20 microns to about 100 microns (e.g., about 20 microns to about 60 microns), and the connection region may have a length L_{con} that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or W_{con1}) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or W_{con1}) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or W_{con1}) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or W_{con1}) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above. Generally, however, the width (W_{con} or W_{con1}) of the proximal opening of the connection region of a sequestration pen is less than the width (W_{ch}) of the microfluidic channel. In some embodiments, the width (W_{con} or W_{con1}) of the proximal opening is about 8%, 9%, 10%, 11%, 12%, 13%, 14%, 15%, 16%, 17%, 18%, 19%, 20%, 22%, 24%, 25%, or 30% of the width (W_{ch}) of the microfluidic channel. That is, the width (W_{ch}) of the microfluidic channel may be at least 2.5 times, 3.0 times, 3.5 times, 4.0 times, 4.5 times, 5.0 times, 6.0 times, 7.0 times, 8.0 times, 9.0 times or at least 10.0 times the width (W_{con} or W_{con1}) of the proximal opening of the connection region of the sequestration pen.

In some embodiments, the size W_{C} (e.g., cross-sectional width Wₑh, diameter, area, or the like) of the channel 122, 322, 618, 718 can be about one and a quarter (1.25), about one and a half (1.5), about two, about two and a half (2.5), about three (3), or more times the size W_{O} (e.g., cross-sectional width W_{con}, diameter, area, or the like) of a chamber opening, e.g., sequestration pen opening 234, 334, and the like. This can reduce the extent of secondary flow and the rate of diffusion (or diffusion flux) through the opening 234, 334 for materials diffusing from a selected chamber (e.g., like sequestration pens 224, 226 of FIG. 2B) into channel 122, 322, 618, 718 and subsequently re-entering a downstream or adjacent chamber (e.g., like sequestration pen 228). The rate of diffusion of a molecule (e.g., an analyte of interest, such as an antibody) is dependent on a number of factors, including (without limitation) temperature, viscosity of the medium, and the coefficient of diffusion D₀ of the molecule. For example, the Do for an IgG antibody in aqueous solution at about 20° C. is about 4.4×10⁻⁷ cm²/sec, while the kinematic viscosity of cell culture medium is about 9×10⁻⁴ m²/sec. Thus, an antibody in cell culture medium at about 20° C. can have a rate of diffusion of about 0.5 microns/sec. Accordingly, in some embodiments, a time period for diffusion from a biological micro-object located within a sequestration pen such as 224, 226, 228, 324 into the channel 122, 322, 618, 718 can be about 10 minutes or less (e.g., about 9, 8, 7, 6, 5 minutes, or less). The time period for diffusion can be manipulated by changing parameters that influence the rate of diffusion. For example, the temperature of the media can be increased (e.g., to a physiological temperature such as about 37° C.) or decreased (e.g., to about 15° C., 10° C., or 4° C.) thereby increasing or decreasing the rate of diffusion, respectively. Alternatively, or in addition, the concentrations of solutes in the medium can be increased or decreased as discussed herein to isolate a selected pen from solutes from other upstream pens.

Accordingly, in some variations, the width (e.g., W_{ch}) of the microfluidic channel at the proximal opening to the connection region of a sequestration pen may be about 50 to 500 microns, about 50 to 300 microns, about 50 to 200 microns, about 70 to 500 microns, about to 70-300 microns, about 70 to 250 microns, about 70 to 200 microns, about 70 to 150 microns, about 70 to 100 microns, about 80 to 500 microns, about 80 to 300 microns, about 80 to 250 microns, about 80 to 200 microns, about 80 to 150 microns, about 90 to 500 microns, about 90 to 300 microns, about 90 to 250 microns, about 90 to 200 microns, about 90 to 150 microns, about 100 to 500 microns, about 100 to 300 microns, about 100 to 250 microns, about 100 to 200 microns, or about 100 to 150 microns. In some embodiments, the width W_{ch} of the microfluidic channel at the proximal opening to the connection region of a sequestration pen may be about 70 to 250 microns, about 80 to 200 microns, or about 90 to 150 microns. The width W_{con} of the opening of the chamber (e.g., sequestration pen) may be about 20 to 100 microns; about 30 to 90 microns; or about 20 to 60 microns. In some embodiments, W_{ch} is about 70-250 microns and W_{con} is about 20 to 100 microns; W_{ch} is about 80 to 200 microns and W_{con} is about 30 to 90 microns; W_{ch} is about 90 to 150 microns, and W_{con} is about 20 to 60 microns; or any combination of the widths of W_{ch} and W_{con} thereof.

In some embodiments, the proximal opening (e.g., 234 or 334) of the connection region of a sequestration pen has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel at the proximal opening, or has a value that lies within a range defined by any two of the foregoing values.

In some embodiments, the width W_{con1} of a proximal opening (e.g., 234 or 334) of a connection region of a sequestration pen may be the same as a width W_{con2} of the distal opening (e.g., 238 or 338) to the isolation region thereof. In some embodiments, the width W_{con1} of the proximal opening may be different than a width W_{con2} of the distal opening, and W_{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., L_{con}) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Ls of about 40-600 microns, about 40-500 microns, about 40-400 microns, about 40-300 microns, about 40-200 microns, about 40-100 microns or about 40-80 microns. The foregoing are examples only, and a sequestration pen may have a length Ls selected to be between any of the values listed above.

According to some embodiments, a sequestration pen may have a specified height (e.g., Hₛ). In some embodiments, a sequestration pen has a height Hₛ of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hₛ of the sequestration pen is typically selected to be the same as the height H_{con} of a connection region and/or the height H_{ch} of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, 20, 22, 24, or 25 microliters/sec. The foregoing are examples only, and the flow of fluidic medium within the microfluidic channel can have a maximum velocity (e.g., Vₘₐₓ) selected to be a value between any of the values listed above. The flow of fluidic medium within the microfluidic channel typically may be flowed at a rate less than the Vₘₐₓ. While the Vₘₐₓ may vary depending on the specific size and numbers of channel and sequestration pens opening thereto, a fluidic medium may be flowed at about 0.1 microliters/sec to about 20 microliters/sec; about 0.1 microliters/sec to about 15 microliters/sec; about 0.1 microliters/sec to about 12 microliters/sec, about 0.1 microliters/sec to about 10 microliters/sec; about 0.1 microliter/sec to about 7 microliters/sec without exceeding the Vₘₐₓ. In some portions of a typical workflow, a flow rate of a fluidic medium may be about 0.1 microliters/sec; about 0.5 microliters/sec; about 1.0 microliters/sec; about 2.0 microliters/sec; about 3.0 microliters/sec; about 4.0 microliters/sec; about 5.0 microliters/sec; about 6.0 microliters/sec; about 7.0 microliters/sec; about 8.0 microliters/sec; about 9.0 microliters/sec; about 10.0 microliters/sec; about 11.0 microliters/sec; or any range defined by two of the foregoing values, e.g., 1-5 microliters/sec or 5-10 microliters/sec. The flow rate of a fluidic medium in the microfluidic channel may be equal to or less than about 12 microliters/sec; about 10 microliters/sec; about 8 microliters/sec, or about 6 microliters/sec.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

Coating solutions and coating agents. In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/expansion/movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG M_{w}<100,000 Da) or alternatively polyethylene oxide (PEO, M_{w}>100,000). In some embodiments, a PEG may have an M_{w} of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules:second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned surface properties. Various factors can alter the physical thickness of the conditioned surface, such as the manner in which the conditioned surface is formed on the substrate (e.g., vapor deposition, liquid phase deposition, spin coating, flooding, and electrostatic coating). In some embodiments, the conditioned surface may have a thickness of about 1 nm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula I, as shown below. Alternatively, the covalently linked coating material may be formed in a two-part sequence, having a structure of Formula II, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including

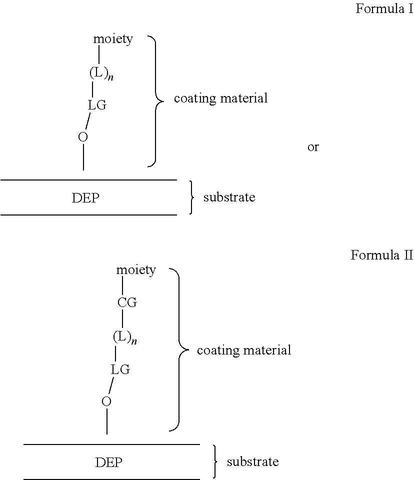

a streptavidin/biotin binding pair, to introduce a protein, peptide, or mixed modified surface.

The coating material may be linked covalently to oxides of the surface of a DEP-configured or EW-configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rₓ and a reactive pairing moiety Rₚₓ (i.e., a moiety configured to react with the reactive moiety Rₓ). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

Further details of suitable coating treatments and modifications, as well as methods of preparation, may be found at U.S. Patent Application Publication No. US2016/0312165 (Lowe, Jr., et al.), U.S. Patent Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic device motive technologies. The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology(ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise DEP electrode activation substrates for selectively inducing motive forces on micro-objects in the fluidic medium 180 in the microfluidic circuit 120 and thereby select, capture, and/or move individual micro-objects or groups of micro-objects.

In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configured devices (e.g., containing optically actuated dielectrophoresis electrode activation substrates) can include those illustrated in U.S. Pat. No. RE 44,711 (Wu, et al.) (originally issued as U.S. Pat. No. 7,612,355), U.S. Pat. No. 7,956,339 (Ohta, et al.), U.S. Pat. No. 9,908,115 (Hobbs et al.), and U.S. Pat. No. 9,403,172 (Short et al), each of which is incorporated herein by reference in its entirety. Examples of suitable OEW configured devices can include those illustrated in U.S. Pat. No. 6,958,132 (Chiou, et al.), and U.S. Pat. No. 9,533,306 (Chiou, et al.), each of which is incorporated herein by reference in its entirety. Examples of suitable optically-actuated electrokinetic devices that include combined OET/OEW configured devices can include those illustrated in U.S. Patent Application Publication No. 2015/0306598 (Khandros, et al.), U.S. Patent Application Publication No 2015/0306599 (Khandros, et al.), and U.S. Patent Application Publication No. 2017/0173580 (Lowe, et al.), each of which is incorporated herein by reference in its entirety.

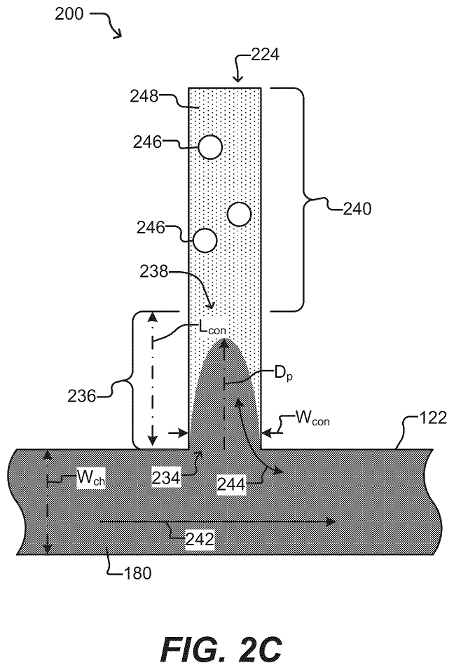

It should be understood that, for purposes of simplicity, the various examples of FIGS. 1-5B may illustrate portions of microfluidic devices while not depicting other portions. Further, FIGS. 1-5B may be part of, and implemented as, one or more microfluidic systems. In one non-limiting example, FIGS. 4A and 4B show a side cross-sectional view and a top cross-sectional view, respectively, of a portion of an enclosure 102 of the microfluidic device 400 having a region/chamber 402, which may be part of a fluidic circuit element having a more detailed structure, such as a growth chamber, a sequestration pen (which may be like any sequestration pen described herein), a flow region, or a flow channel. For instance, microfluidic device 400 may be similar to microfluidic devices 100, 175, 200, 300, 520 or any other microfluidic device as described herein. Furthermore, the microfluidic device 400 may include other fluidic circuit elements and may be part of a system including control and monitoring equipment 152, described above, having one or more of the media module 160, motive module 162, imaging module 164, optional tilting module 166, and other modules 168. Microfluidic devices 175, 200, 300, 520 and any other microfluidic devices described herein may similarly have any of the features described in detail for FIGS. 1A-1B and 4A-4B.

As shown in the example of FIG. 4A, the microfluidic device 400 includes a support structure 104 having a bottom electrode 404 and an electrode activation substrate 406 overlying the bottom electrode 404, and a cover 110 having a top electrode 410, with the top electrode 410 spaced apart from the bottom electrode 404. The top electrode 410 and the electrode activation substrate 406 define opposing surfaces of the region/chamber 402. A fluidic medium 180 contained in the region/chamber 402 thus provides a resistive connection between the top electrode 410 and the electrode activation substrate 406. A power source 412 configured to be connected to the bottom electrode 404 and the top electrode 410 and create a biasing voltage between the electrodes, as required for the generation of DEP forces in the region/chamber 402, is also shown. The power source 412 can be, for example, an alternating current (AC) power source.

In certain embodiments, the microfluidic device 200 illustrated in FIGS. 4A and 4B can have an optically-actuated DEP electrode activation substrate. Accordingly, changing patterns of light 418 from the light source 416, which may be controlled by the motive module 162, can selectively activate and deactivate changing patterns of DEP electrodes at regions 414 of the inner surface 408 of the electrode activation substrate 406. (Hereinafter the regions 414 of a microfluidic device having a DEP electrode activation substrate are referred to as “DEP electrode regions.”) As illustrated in FIG. 4B, a light pattern 418 directed onto the inner surface 408 of the electrode activation substrate 406 can illuminate select DEP electrode regions 414a (shown in white) in a pattern, such as a square. The non-illuminated DEP electrode regions 414 (cross-hatched) are hereinafter referred to as “dark” DEP electrode regions 414. The relative electrical impedance through the DEP electrode activation substrate 406 (i.e., from the bottom electrode 404 up to the inner surface 408 of the electrode activation substrate 406 which interfaces with the fluidic medium 180 in the flow region 106) is greater than the relative electrical impedance through the fluidic medium 180 in the region/chamber 402 (i.e., from the inner surface 408 of the electrode activation substrate 406 to the top electrode 410 of the cover 110) at each dark DEP electrode region 414. An illuminated DEP electrode region 414a, however, exhibits a reduced relative impedance through the electrode activation substrate 406 that is less than the relative impedance through the fluidic medium 180 in the region/chamber 402 at each illuminated DEP electrode region 414a.

With the power source 412 activated, the foregoing DEP configuration creates an electric field gradient in the fluidic medium 180 between illuminated DEP electrode regions 414a and adjacent dark DEP electrode regions 414, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 414 at the inner surface 408 of the region/chamber 402 by changing light patterns 418 projected from a light source 416 into the microfluidic device 400. Whether the DEP forces attract or repel nearby micro-objects can depend on such parameters as the frequency of the power source 412 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 420 of illuminated DEP electrode regions 414a illustrated in FIG. 4B is an example only. Any pattern of the DEP electrode regions 414 can be illuminated (and thereby activated) by the pattern of light 418 projected into the microfluidic device 400, and the pattern of illuminated/activated DEP electrode regions 414 can be repeatedly changed by changing or moving the light pattern 418.

In some embodiments, the electrode activation substrate 406 can comprise or consist of a photoconductive material. In such embodiments, the inner surface 408 of the electrode activation substrate 406 can be featureless. For example, the electrode activation substrate 406 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 414 can be created anywhere and in any pattern on the inner surface 408 of the electrode activation substrate 406, in accordance with the light pattern 418. The number and pattern of the DEP electrode regions 214 thus need not be fixed, but can correspond to the light pattern 418. Examples of microfluidic devices having a DEP configuration comprising a photoconductive layer such as discussed above have been described, for example, in U.S. Pat. No. RE 44,711 (Wu, et al.) (originally issued as U.S. Pat. No. 7,612,355), each of which is incorporated herein by reference in its entirety.

In other embodiments, the electrode activation substrate 406 can comprise a substrate comprising a plurality of doped layers, electrically insulating layers (or regions), and electrically conductive layers that form semiconductor integrated circuits, such as is known in semiconductor fields. For example, the electrode activation substrate 406 can comprise a plurality of phototransistors, including, for example, lateral bipolar phototransistors, with each phototransistor corresponding to a DEP electrode region 414. Alternatively, the electrode activation substrate 406 can comprise electrodes (e.g., conductive metal electrodes) controlled by phototransistor switches, with each such electrode corresponding to a DEP electrode region 414. The electrode activation substrate 406 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 414 at the inner surface 408 of the electrode activation substrate 406 and the bottom electrode 404, and those electrical connections (i.e., phototransistors or electrodes) can be selectively activated and deactivated by the light pattern 418, as described above.

Examples of microfluidic devices having electrode activation substrates that comprise phototransistors have been described, for example, in U.S. Pat. No. 7,956,339 (Ohta et al.) and U.S. Pat. No. 9,908,115 (Hobbs et al.), the entire contents of each of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 410 is part of a first wall (or cover 110) of the enclosure 402, and the electrode activation substrate 406 and bottom electrode 404 are part of a second wall (or support structure 104) of the enclosure 102. The region/chamber 402 can be between the first wall and the second wall. In other embodiments, the electrode 410 is part of the second wall (or support structure 104) and one or both of the electrode activation substrate 406 and/or the electrode 410 are part of the first wall (or cover 110). Moreover, the light source 416 can alternatively be used to illuminate the enclosure 102 from below.

With the microfluidic device 400 of FIGS. 4A-4B having a DEP electrode activation substrate, the motive module 162 of control and monitoring equipment 152, as described for FIG. 1A herein, can select a micro-object (not shown) in the fluidic medium 180 in the region/chamber 402 by projecting a light pattern 418 into the microfluidic device 400 to activate a first set of one or more DEP electrodes at DEP electrode regions 414a of the inner surface 408 of the electrode activation substrate 406 in a pattern (e.g., square pattern 420) that surrounds and captures the micro-object. The motive module 162 can then move the in situ-generated captured micro-object by moving the light pattern 418 relative to the microfluidic device 400 to activate a second set of one or more DEP electrodes at DEP electrode regions 414. Alternatively, the microfluidic device 400 can be moved relative to the light pattern 418.

In other embodiments, the microfluidic device 400 may be a DEP configured device that does not rely upon light activation of DEP electrodes at the inner surface 408 of the electrode activation substrate 406. For example, the electrode activation substrate 406 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 414, thereby creating a net DEP force on a micro-object (not shown) in region/chamber 402 in the vicinity of the activated DEP electrodes. Depending on such characteristics as the frequency of the power source 412 and the dielectric properties of the medium (not shown) and/or micro-objects in the region/chamber 402, the DEP force can attract or repel a nearby micro-object. By selectively activating and deactivating a set of DEP electrodes (e.g., at a set of DEP electrodes regions 414 that forms a square pattern 420), one or more micro-objects in region/chamber 402 can be selected and moved within the region/chamber 402. The motive module 162 in FIG. 1A can control such switches and thus activate and deactivate individual ones of the DEP electrodes to select, and move particular micro-objects (not shown) around the region/chamber 402. Microfluidic devices having a DEP electrode activation substrate that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

Regardless of whether the microfluidic device 400 has a dielectrophoretic electrode activation substrate, an electrowetting electrode activation substrate or a combination of both a dielectrophoretic and an electrowetting activation substrate, a power source 412 can be used to provide a potential (e.g., an AC voltage potential) that powers the electrical circuits of the microfluidic device 400. The power source 412 can be the same as, or a component of, the power source 192 referenced in FIG. 1A. Power source 412 can be configured to provide an AC voltage and/or current to the top electrode 410 and the bottom electrode 404. For an AC voltage, the power source 412 can provide a frequency range and an average or peak power (e.g., voltage or current) range sufficient to generate net DEP forces (or electrowetting forces) strong enough to select and move individual micro-objects (not shown) in the region/chamber 402, as discussed above, and/or to change the wetting properties of the inner surface 408 of the support structure 104 in the region/chamber 202, as also discussed above. Such frequency ranges and average or peak power ranges are known in the art. See, e.g., U.S. Pat. No. 6,958,132 (Chiou, et al.), U.S. Pat. No. RE44,711 (Wu, et al.) (originally issued as U.S. Pat. No. 7,612,355), and U.S. Patent Application Publication Nos. 2014/0124370 (Short, et al.), 2015/0306598 (Khandros, et al.), 2015/0306599 (Khandros, et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger, et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger, et al.), which is incorporated herein by reference in its entirety. Use of gravity (e.g., by tilting the microfluidic device and/or the support to which the microfluidic device is attached) may be useful for bulk movement of cells into or out of the sequestration pens from/to the flow region. Magnetic forces may be employed to move micro-objects including paramagnetic materials, which can include magnetic micro-objects attached to or associated with a biological micro-object. Alternatively, or in additional, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in International Patent Publication No. WO2017/117408 (Kurz, et al.), which is incorporated herein by reference in its entirety.

In some embodiments, DEP forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP forces can be applied prior to the other forces. In other embodiments, the DEP forces can be applied after the other forces. In still other instances, the DEP forces can be applied in an alternating manner with the other forces. For the microfluidic devices described herein, repositioning of micro-objects may not generally rely upon gravity or hydrodynamic forces to position or trap micro-objects at a selected position. Gravity may be chosen as one form of repositioning force, but the ability to reposition of micro-objects within the microfluidic device does not rely solely upon the use of gravity. While fluid flow in the microfluidic channels may be used to introduce micro-objects into the microfluidic channels (e.g., flow region), such regional flow is not relied upon to pen or unpen micro-objects, while localized flow (e.g., force derived from actuating a deformable surface) may, in some embodiments, be selected from amongst the other types of repositioning forces described herein to pen or unpen micro-objects or to export them from the microfluidic device.

When DEP is used to reposition micro-objects, bulk fluidic flow in a channel is generally stopped prior to applying DEP to micro-objects to reposition the micro-objects within the microfluidic circuit of the device, whether the micro-objects are being repositioned from the channel into a sequestration pen or from a sequestration pen into the channel. Bulk fluidic flow may be resumed thereafter.

System. Returning to FIG. 1A, a system 150 for operating and controlling microfluidic devices is shown, such as for controlling the microfluidic device 100. The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂ (or higher). The media module 160 may also control the temperature of an enclosure of the media source, for example, to provide feeder cells in the media source with proper temperature control.

Motive module. The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an optoelectrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configured device may include one or more electrodes that apply a non-uniform electric field in the microfluidic circuit 120 sufficient to exert a dielectrophoretic force on micro-objects in the microfluidic circuit 120. An OET configured device may include photo-activatable electrodes to provide selective control of movement of micro-objects in the microfluidic circuit 120 via light-induced dielectrophoresis.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescent label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g., for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g., a high-pressure mercury lamp) or a Xenon arc lamp. The imaging device may further include a microscope (or an optical train), which may or may not include an eyepiece.

Support Structure. System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e., at 0° relative to x- and y-axes), a vertical orientation (i.e., at 900 relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) to 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 65°, 70°, 75°, 80°, 90° relative to the x-axis or any degree therebetween. When the microfluidic device is tilted at angles greater than about 15, tilting may be performed to create bulk movement of micro-objects into/out of sequestration pens from/into the flow region (e.g., microfluidic channel). In some embodiments, the support structure 190 can hold the microfluidic device 100 at a fixed angle of 0.1°, 0.2°, 0.3°, 0.4°, 0.5°, 0.6°, 0.7°, 0.8°, 0.9°, 1°, 2°, 3°, 4°, 5°, or 10° relative to the x-axis (horizontal), so long as DEP is an effective force to move micro-objects out of the sequestration pens into the microfluidic channel. Since the surface of the electrode activation substrate is substantially flat, DEP forces may be used even when the far end of the sequestration pen, opposite its opening to the microfluidic channel, is disposed at a position lower in a vertical direction than the microfluidic channel.

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e., an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 5°, about 4°, about 3° or less than about 2° relative to the x-axis (horizontal), thereby placing the sequestration pens at a lower potential energy relative to the flow path. In some other embodiments, when long term culturing (e.g., for more than about 2, 3, 4, 5, 6, 7 or more days) is performed within the microfluidic device, the device may be supported on a culturing support and may be tilted at a greater angle of about 10°, 15°, 20°, 25°, 30°, or any angle therebetween to retain biological micro-objects within the sequestration pens during the long-term culturing period. At the end of the culturing period, the microfluidic device containing the cultured biological micro-objects may be returned to the support 190 within system 150, where the angle of tilting is decreased to values as described above, affording the use of DEP to move the biological micro-objects out of the sequestration pens. Further examples of the use of gravitational forces induced by tilting are described in U.S. Pat. No. 9,744,533 (Breinlinger et al.), the contents of which are herein incorporated by reference in its entirety.

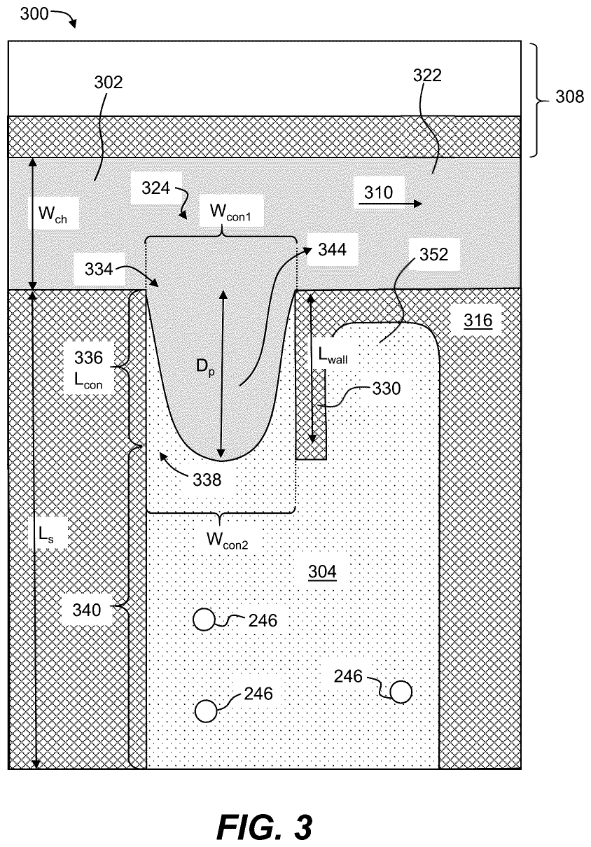

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electro-wetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 502 mounted on PCBA 522, as well.

In some embodiments, the nest 500 can comprise an electrical signal generation subsystem 504 configured to measure the amplified voltage at the microfluidic device 520 and then adjust its own output voltage as needed such that the measured voltage at the microfluidic device 520 is the desired value. In some embodiments, the waveform amplification circuit can have a +6.5V to −6.5V power supply generated by a pair of DC-DC converters mounted on the PCBA 322, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 3A the controller 308 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). In the embodiment illustrated in FIG. 5A, the support structure 500 comprises an inlet 516 and an outlet 518 to receive cooled fluid from an external reservoir (not shown) of the cooling unit, introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical sub-system. FIG. 5B is a schematic of an optical sub-system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560 includes a mirror, the modulator can have a plurality of mirrors. Each mirror of the plurality of mirrors can have a size of about 5 microns×5 microns to about 10 microns×10 microns, or any values therebetween. The structured light modulator 560 can include an array of mirrors (or pixels) that is 2000×1000, 2580×1600, 3000×2000, or any values therebetween. In some embodiments, only a portion of an illumination area of the structured light modulator 560 is used. The structured light modulator 560 can transmit the selected subset of light to a first dichroic beam splitter 558, which can reflect this light to a first tube lens 562.

The first tube lens 562 can have a large clear aperture, for example, a diameter larger than about 40 mm to about 50 mm, or more, providing a large field of view. Thus, the first tube lens 562 can have an aperture that is large enough to capture all (or substantially all) of the light beams emanating from the structured light modulator 560.

The structured light 515 having a wavelength of about 400 nm to about 710 nm, may alternatively or in addition, provide fluorescent excitation illumination to the microfluidic device.

The second light source 554 may provide unstructured brightfield illumination. The brightfield illumination light 525 may have any suitable wavelength, and in some embodiments, may have a wavelength of about 400 nm to about 760 nm. The second light source 554 can transmit light to a second dichroic beam splitter 564 (which also may receive illumination light 535 from the third light source 556), and the second light, brightfield illumination light 525, may be transmitted therefrom to the first dichroic beam splitter 558. The second light, brightfield illumination light 525, may then be transmitted from the first dichroic beam splitter 558 to the first tube lens 562.

The third light source 556 can transmit light through a matched pair relay lens (not shown) to a mirror 566. The third illumination light 535 may therefrom be reflected to the second dichroic beam splitter 5338 and be transmitted therefrom to the first beam splitter 5338, and onward to the first tube lens 5381. The third illumination light 535 may be a laser and may have any suitable wavelength. In some embodiments, the laser illumination 535 may have a wavelength of about 350 nm to about 900 nm. The laser illumination 535 may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U. S. Application Publication Nos. 2017/0165667 (Beaumont, et al.) and 2018/0298318 (Kurz, et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The light from the first, second, and third light sources (552, 554, 556) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500, as described in FIG. 5A, can be integrated with the optical apparatus 510 and be a part of the apparatus 510. The nest 500 can provide electrical connection to the enclosure and be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective. The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 1350. The objective lens 570 can have one or more magnification levels available such as, 4×, 10×, 20×.

Modes of illumination. In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, similarly as described for FIGS. 4A-4B, which can be moved and adjusted. The optical apparatus 560 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to thereby increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots, which may form an illumination pattern, into each of the plurality of sequestration pens in the sample plane 574 within the field of view. For example, each of the plurality of illumination spots can have a size of about 5 microns×5 microns; 10 microns×10 microns; 10 microns×30 microns, 30 microns×60 microns, 40 microns×40 microns, 40 microns×60 microns, 60 microns×120 microns, 80 microns×100 microns, 100 microns×140 microns and any values there between. The illumination spots may individually have a shape that is circular, square, or rectangular. Alternatively, the illumination spots may be grouped within a plurality of illumination spots (e.g., an illumination pattern) to form a larger polygonal shape such as a rectangle, square, or wedge shape. The illumination pattern may enclose (e.g., surround) an unilluminated space that may be square, rectangular or polygonal. For example, each of the plurality of illumination spots can have an area of about 150 to about 3000, about 4000 to about 10000, or 5000 to about 15000 square microns. An illumination pattern may have an area of about 1000 to about 8000, about 4000 to about 10000, 7000 to about 20000, 8000 to about 22000, 10000 to about 25000 square microns and any values there between.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U. S. Application Publication No. 2016/0160259 (Du); U.S. Pat. No. 9,996,920 (Du et al.); and International Application Publication No. WO2017/102748 (Kim, et al.). The optical system 510 may also be employed in assay methods to determine concentrations of reagents/assay products, and further details are found in U.S. Pat. No. 8,921,055 (Chapman), U.S. Pat. No. 10,010,882 (White et al.), and U.S. Pat. No. 9,889,445 (Chapman et al.); International Application Publication No. WO2017/181135 (Lionberger, et al.); and International Application Serial No. PCT/US2018/055918 (Lionberger, et al.). Further details of the features of optical apparatuses suitable for use within a system for observing and manipulating micro-objects within a microfluidic device, as described herein, may be found in WO2018/102747 (Lundquist, et al), the disclosure of which is herein incorporated by reference in its entirety.

Additional system components for maintenance of viability of cells within the sequestration pens of the microfluidic device. In order to promote growth and/or expansion of cell populations, environmental conditions conducive to maintaining functional cells may be provided by additional components of the system. For example, such additional components can provide nutrients, cell growth signaling species, pH modulation, gas exchange, temperature control, and removal of waste products from cells.

The following Examples illustrate these methods for assaying micro-objects in a microfluidic device.

### Example 1

System and Microfluidic device: The following experiments were performed using an OptoSelect™ microfluidic (or nanofluidic) device manufactured by Berkeley Lights, Inc. and controlled by an optical instrument which was also manufactured by Berkeley Lights, Inc. The instrument included: a mounting stage for the microfluidic device coupled to a temperature controller; a pump and fluid medium conditioning component; an optical train including a camera and a structured light source suitable for activating phototransistors within the microfluidic device; and software for controlling the instrument, including performing image analysis and automated detection and repositioning of micro-objects. The OptoSelect™ device included a substrate configured with OptoElectroPositioning (OEP™) technology, which provides a phototransistor-activated dielectrophoresis (DEP) force. The device also included a plurality of microfluidic channels, each having a plurality of NanoPen™ chambers (or sequestration pens) fluidically connected thereto. The volume of each sequestration pen was around 1×10⁶ cubic microns. The microfluidic device included conditioned interior surfaces, which are described in U.S. Patent Application Publication No. US2016/0312165 (Lowe, Jr., et al.), International Patent Application Publication WO2017/205830 (Lowe, Jr., et al.), and International Patent Application Publication WO2019/01880 (Beemiller et al.), each of which disclosures is herein incorporated by reference in its entirety.

Priming regime: 250 microliters of 100% carbon dioxide was flowed in at a rate of 12 microliters/sec. This was followed by 250 microliters of a wetting solution including surface conditioning reagents to provide conditioned surfaces as described in the referenced publications above.

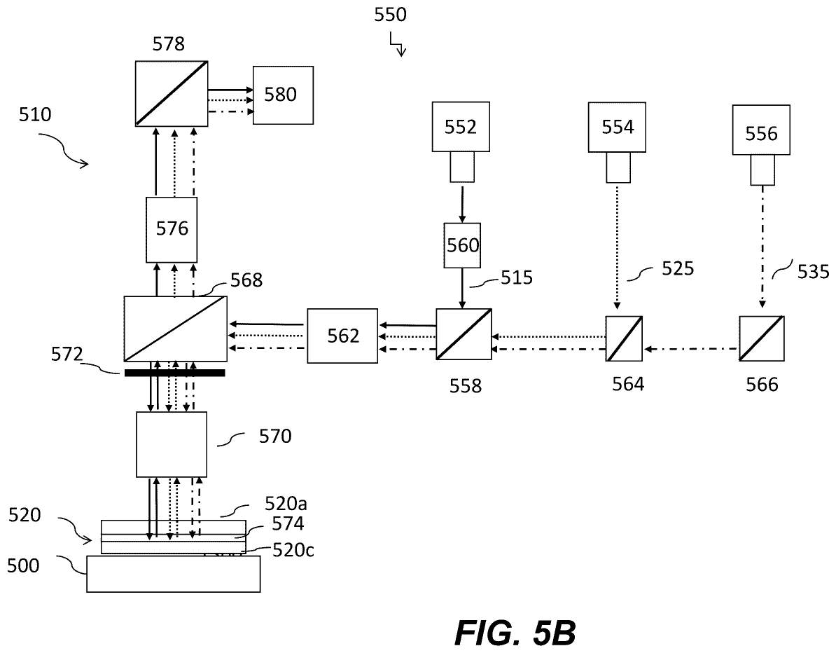

50 μL of a first fluidic medium containing 4×AOPI dye was imported into the microfluidic device until the channel was filled. The AOPI dye was allowed to diffuse into the chambers and to equilibrate for 30 minutes. An image was periodically obtained, e.g., at 5 minutes and 10 minutes during the equilibration and the concentration of AOPI dye (represented by the intensity of the fluorescence detected) was recorded to track the process of the diffusion of the AOPI dye into the chambers to reach a substantially uniform concentration. FIG. 8 shows the intensity gradients of the AO dye and the PI dye in the microfluidic devices at time 0, 5 minutes, 10 minutes, and 15 minutes. As can be seen in FIG. 8, 5 minutes is sufficient for the AOPI dye in the first fluidic medium to diffuse into the chambers and substantially reach equilibrium across the flow region (microfluidic channel) and the chambers. In the gradient curve, the x-axis is the vertical displacement across the chamber in the unit of pixel and the y-axis is the intensity of the fluorescence signal.

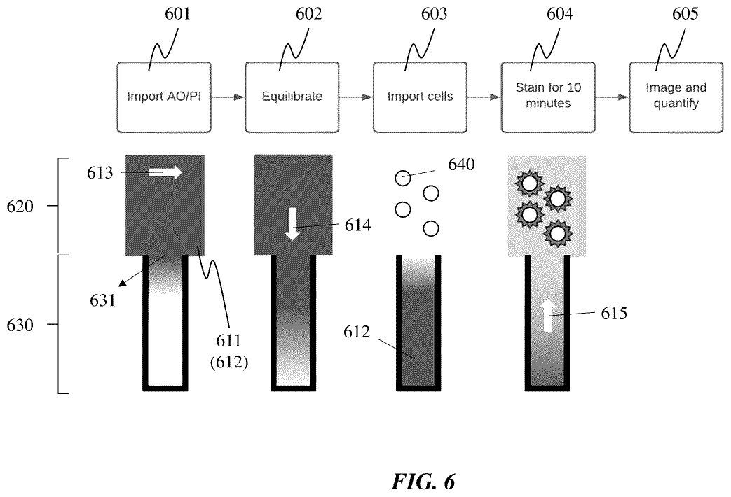

A second fluidic medium (25 μL) containing cells (Pan T cells) were imported into the microfluidic device until it filled the channel. The AOPI dye retained in the chamber was then allowed to diffuse out of the chamber and into the microfluidic channel for 18 minutes and stain the cells. An image was taken every 3 minutes and the concentration of AOPI dye (represented by the intensity of the fluorescence detected) was recorded to track the process of the diffusion of the AOPI dye into the channels from the chambers and stained the cells. The light intensity of the cells increased in the first 3 minutes and then plateaued. There was no observable spatial-dependent intensity variation across the channel after 5 minutes (figure not shown). Thus, it appeared that 5 minutes should be sufficient for cell staining. Then, the images were combined, and the background signal was removed to obtain composite images as shown in FIG. 9. FIG. 9 shows two rows of microfluidic channel 910 and chambers 920 open laterally to the microfluidic channel 910. The T cells 901 were stained by the AOPI dye within the microfluidic channel 910 while the dye was diffusing out of the chamber 920. According to FIG. 9, the percentage of viable cells was about 90% (the cells pointed to by the arrows in this figure were dead cells).

### Example 2

CAR T cells were prepared using the following process. Frozen T cells were thawed and re-suspended in 20 mL CTL (2.89×10⁶ cells/mL). The T cells were than activated by incubation with anti-CD3/CD28 beads (5.78×10⁷) overnight. The activated T cells were then seeded on a 96 well Flat Bottom TC treated plate (5×10⁴ per well). The cells were then transduced using a 47.5 uL/well of viral suspension (Sirion Biotech, Catalog #78600 Lot #210513 Titer—5.0×10⁵ IFU/uL, MOI 50) and a 2.5 uL/well of LentiBOOST® (Sirion Biotech) and cultured at 37 degrees Celsius. After this culture, the cells were collected and re-suspended at a density of 11321/2.5p L=4.53×10⁶/mL.

The following 4 dyes (e.g., reagents) were selected as the staining material: (1) 4 μL of FMC63 (CAR)-FITC (FITC cube); (2) 4 μL (with a density of 200 μg/mL) of CD3-AF647 (CY5 cube); (3) 4 μL (with a density of 50 μg/mL) of CD8-PE/Dazzle 594 (TRED cube); and (4) 4 μL (with a density of 100 μg/mL) of CD25-BV421 (DAPI cube). 4 μL of a human TruStain FcX™ (as a Fc Receptor Blocking Solution) was also used. These 5 reagents were then mixed with 30 μL of a FACS buffer solution (1X DPBS w/o Ca2+ and Mg2+; 2% FBS; 5 mM EDTA; 10 mM HEPES) containing so that the reagents are 4 times more concentrated than the recommended working concentration, thereby creating the first fluidic medium.

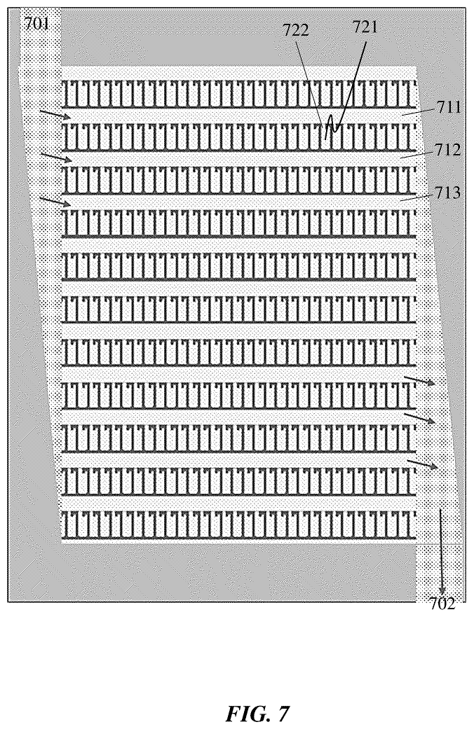

50 μL of this first fluidic medium was imported into the microfluidic device until the channel was filled. The dyes were allowed to diffuse into the chambers and to equilibrate for 60 minutes. 25 μL of a second fluidic medium containing the CAR T cells was imported into the microfluidic device until it filled the channels. The dyes retained in the chamber were then allowed to diffuse out of the chamber and into the microfluidic channel for 60 minutes and stained the cells. An image was taken every 20 minutes to track the diffusion of the 4 reagents into the channel from the chambers and staining of the cells (figures not shown). The images were adjusted by removing the background signal to show the cells stained (FIG. 10). Positive and negative stained cells were able to be determined using Image Analyzer with high accuracy verified by visual inspection. The Image Analyzer has been described, for example, in WO2018102748 (Kim et al,) filed on Dec. 1, 2017, the entire disclosure of which is incorporated herein by reference. With total 11321 cells counted; the percentages of cells stained by the four dyes respectively can be calculated as below. The staining of CD25 was less obvious in FIG. 10, but two positively stained cells were pointed by arrows as examples.
CD3 (CY5): 10608/11321=93.7%
CD8 (TRED): 4200/11321=37.1%
CD25 (DAPI): 1359/11321=12.0%
CAR (FITC): 2472/11321=21.8%

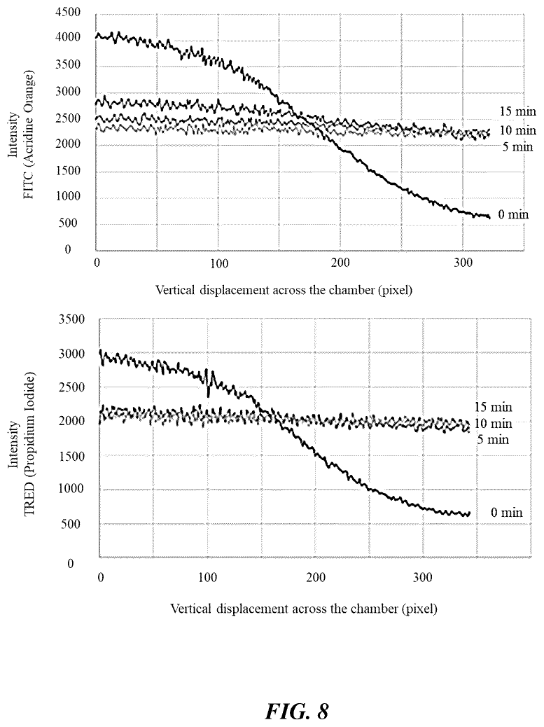

Moreover, in the cells positively stained by CD3, the percentage of cells that were positively stained by both CD3 and CAR (FMC63), the percentage of cells that were positively stained by both CD3 and CD8, and the percentage of cells that were positively stained by CD3, CAR (FMC63), and CD8 were calculated. The calculation and data shown in FIG. 11 indicate that the percentages obtained using FACS and the method of the present disclosure (i.e., on-chip measurement) were consistent, suggesting the method of the present disclosure is reliable in assaying cells:
(CAR∩CD3)/CD3: 2161/10608=20.3%
   FACS: 20.2% (Day 8); 25.7% (Day 10)
(CD8∩CD3)/CD3: 4096/10608=38.6%
   FACS: 41.1% (Day 8); 34.2% (Day 10)
(CAR∩(CD8∩CD3))/(CD8∩CD3): 462/4096=11.3%
   FACS: 10.4% (Day 8); 16.8% (Day 10)

In certain embodiments, the disclosure further provides machine-readable storage devices for storing non-transitory machine-readable instructions for carrying out the foregoing methods. The machine-readable instructions can further control the imaging device used to obtain the images.

Although specific embodiments and applications of the disclosure have been described in this specification, these embodiments and applications are exemplary only, and many variations are possible.

### List of Some Embodiments of the Disclosure

Embodiment 1: A method of contacting a micro-object with a reagent within a microfluidic device, wherein the microfluidic device comprises a microfluidic circuit material defining a flow region and a chamber comprising a proximal opening fluidically connecting the chamber to the flow region; the method comprises: introducing a micro-object into the flow region of the microfluidic device; and allowing a reagent to diffuse from the chamber to the flow region and to contact the micro-object.

Embodiment 2: The method of claim 1, wherein the chamber comprises an unswept region.

Embodiment 3: The method of claim 1, wherein the chamber comprises an isolation region and a connection region fluidically connecting the isolation region to the flow region, wherein the isolation region is an unswept region of the microfluidic device.

Embodiment 4: The method of claim 2 or claim 3, wherein the reagent is present within the unswept region of the microfluidic device.

Embodiment 5: The method of any one of claim 1 to 4, wherein the reagent is present in the chamber before introducing the micro-object into the flow region.

Embodiment 6: The method of any one of claims 1 to 5, wherein the reagent is present in the chamber by: introducing a first fluidic medium comprising the reagent to the flow region of the microfluidic device; and allowing the reagent to diffuse from the flow region into the chamber.

Embodiment 7: The method of claim 6, wherein the reagent is allowed to diffuse from the flow region into the chamber for at least about 0.5, 1, 3, 5, 10, 20, 40, 60, 90, 120, 150, 200, or 300 minutes.

Embodiment 8: The method of claim 6 or claim 7, wherein allowing the reagent to diffuse from the flow region into the chamber comprises maintaining a continuous perfusion of the first fluidic medium comprising the reagent.

Embodiment 9: The method of any one of claims 6 to 8, wherein the first fluidic medium comprises the reagent at an initial concentration, and further wherein the initial concentration is at least about 1, about 2, about 4, about 8, about 12, about 16, about 20, about 40, about 100, about 200, or about 2000 times higher than a working concentration of the reagent permitting an interaction between the reagent and the micro-object.

Embodiment 10: The method of any one of claims 6 to 9, wherein allowing the reagent to diffuse from the flow region into the chamber comprises allowing the reagent to equilibrate between the flow region and the chamber.

Embodiment 11: The method of any one of claims 1 to 10, wherein the reagent is a mixture of assay reagents.

Embodiment 12: The method of any one of claims 1 to 11, wherein introducing the micro-object comprises introducing a second fluidic medium comprising the micro-object, and stopping a flow of the second fluidic medium after the micro-object is within the flow region.

Embodiment 13: The method of any one of claims 1 to 12, wherein the micro-object substantially has no contact with the reagent until the reagent diffuses from the chamber to the flow region and to contact the micro-object.

Embodiment 14: The method of any one of claims 1 to 13, wherein introducing the micro-object comprises introducing the micro-object to a position within the flow region that is proximal to the opening of the chamber.

Embodiment 15: The method of any one of claims 1 to 14, wherein allowing the reagent to diffuse from the chamber to the flow region comprises contacting the micro-object with the reagent within the flow region.

Embodiment 16: The method of any one of claims 1 to 15, wherein allowing the reagent to diffuse from the chamber to the flow region to contact the micro-object within the flow region comprises allowing the reagent to diffuse for at least about 0.5, 1, 3, 5, 10, 20, 40, 60, 90, 120, 150, 200, or 300 minutes.

Embodiment 17: The method of any one of claims 1 to 16, wherein allowing the reagent to diffuse from the chamber to the flow region to contact the micro-object within the flow region comprises allowing the reagent to equilibrate between the flow region and the chamber.

Embodiment 18: The method of any one of claims 1 to 17, further comprising introducing a prepolymer composition into the flow region, wherein the prepolymer composition is configured to form an in situ-generated structure within the microfluidic device.

Embodiment 19: The method of claim 18, wherein introducing a prepolymer composition into the flow region comprises introducing a third fluidic medium comprising the prepolymer composition into the flow region; and allowing the prepolymer composition to diffuse into the chamber.

Embodiment 20: The method of claim 19, wherein the prepolymer composition is soluble in the third fluidic medium.

Embodiment 21: The method of any one of claims 18 to 20, further comprising activating solidification of the prepolymer composition, thereby forming the in situ-generated structure within the microfluidic device.

Embodiment 22: The method of claim 21, wherein the in situ-generated structure is formed within the chamber, within the flow region, or both.

Embodiment 23: The method of claim 21 or claim 22, wherein the in situ-generated structure is formed in an area proximal to the proximal opening of the chamber.

Embodiment 24: The method of any one of claims 18 to 23, wherein the in situ-generated structure has a porosity that restricts passage of the reagent.

Embodiment 25: The method of claim 24, wherein the porosity blocks passage of the reagent.

Embodiment 26: The method of any one of claims 18 to 25, wherein the in situ-generated structure seals the proximal opening of the chamber.

Embodiment 27: The method of claim 26, further comprising unsealing the proximal opening of the chamber.

Embodiment 28: The method of claim 27, wherein unsealing the proximal opening of the chamber comprises removing or reducing the size of the in situ-generated structure so that the proximal opening of the chamber is no longer sealed by the in situ-generated structure.

Embodiment 29: The method of any one of claims 18 to 28, wherein the prepolymer composition comprises: a first polyethylene glycol polymer molecule and a second polyethylene glycol polymer molecule, each comprising a respective polyethylene glycol moiety and a covalently linked reactive moiety Rₓ; a crosslinker molecule comprising a first reactive moiety R_{xP} disposed at a first end of a linker moiety L and a second reactive moiety R_{xP} disposed at a second end of the linker moiety L, wherein each of the first and the second crosslinker moiety R_{xP} is configured to be activatable to react with a respective reactive moiety Rₓ of the first and the second polyethylene polymer molecules, optionally wherein each of the first and the second polyethylene polymer molecules comprises different polyethylene glycol moieties or each comprises a same polyethylene glycol moiety.

Embodiment 30: The method of claim 29, wherein the first polyethylene glycol moiety comprises a 1-arm, 2-arm, 4-arm or 8-arm polyethylene glycol moiety.

Embodiment 31: The method of claim 29 or claim 30, wherein the first polyethylene glycol moiety has a molecular weight from about 500 Da to about 25K Da.

Embodiment 32: The method of any one of claims 29 to 31, wherein the second polyethylene glycol moiety comprises a 1-arm, 2-arm, 4-arm or 8-arm polyethylene glycol moiety.

Embodiment 33: The method of any one of claims 29 to 32, wherein the second polyethylene glycol moiety has a molecular weight from about 500 Da to about 25K Da.

Embodiment 34: The method of any one of claims 29 to 33, wherein the first polyethylene glycol moiety has a molecular weight of about 10K Da and the second polyethylene glycol moiety has a molecular weight of about 20K Da.

Embodiment 35: The method of any one of claims 29 to 34, wherein the first polyethylene glycol molecule and the second polyethylene molecule are present in the composition in a ratio from about 1:100 to about 100:1.

Embodiment 36: The method of claim 35, wherein the ratio of the first polyethylene glycol molecule and the second polyethylene molecule is about 1:1; about 1:2; about 1:3; about 1:4; about 1:5; about 1:6; about 1:7; about 1:8; about 1:9; about 1:10; about 10:1; about 9:1; about 8:1; about 7:1; about 6:1; about 5:1; about 4:1; about 3:1; or about 2:1.

Embodiment 37: The method of any one of claims 29 to 36, wherein the crosslinker molecule comprises a vic-diol.

Embodiment 38: The method of any one of claims 29 to 36, wherein the crosslinker comprises a peptide sequence configured to be a substrate to a protease.

Embodiment 39: The method of claim 38, wherein the crosslinker further comprises a first thiol moiety at a first end of the crosslinker, and a second thiol moiety at the second end of the crosslinker, each configured to react with a norbornenyl moiety of the first or the second polyethylene glycol polymer molecule.

Embodiment 40: The method of claim 38 or claim 39, wherein the peptide sequence comprises GCRDLPRTGGDRCG.

Embodiment 41: The method of any one of claims 38 to 40, wherein the crosslinker comprises a peptide sequence configured to be a tryptase substrate.

Embodiment 42: The method of any one of claims 29 to 36, wherein the crosslinker has a formula: HS-LB4-SH (Formula V); wherein linker backbone LB4 comprises 3 to 200 non-hydrogen atoms selected from any combination of silicon, carbon, nitrogen, oxygen, sulfur and phosphorus atoms.

Embodiment 43: The method of claim 42, wherein the crosslinker is Sodium 2,3-dimercaptopropanesulfonate monohydrate.

Embodiment 44: The method of any one of claims 29 to 43, further comprising an inhibitor configured to inhibit reaction of the crosslinker with the reactive moiety of the first and/or the second polyethylene glycol polymer molecule.

Embodiment 45: The method of claim 44, wherein the inhibitor is sodium ascorbate, MEHQ, or 4-hydroxy TEMPO.

Embodiment 46: The method of any one of claims 1 to 45, further comprising observing an interaction between the reagent and the micro-object.

Embodiment 47: The method of claim 46, wherein observing an interaction between the reagent and the micro-object comprises detecting a first signal associated with the reagent.

Embodiment 48: The method of claim 46 or claim 47, wherein observing an interaction between the reagent and the micro-object comprises observing a morphologic change of the micro-object.

Embodiment 49: The method of any one of claims 46 to 48, wherein observing an interaction between the reagent and the micro-object comprises detecting a second signal associated with a reporter reagent, wherein the reporter reagent is introduced together with the micro-object.

Embodiment 50: The method of any one of claims 46 to 48, wherein observing an interaction between the reagent and the micro-object comprises detecting a second signal associated with a reporter reagent, wherein the reporter reagent is present in the chamber when the micro-object is introduced.

Embodiment 51: The method of claim 50, wherein the reporter reagent diffuses faster than the reagent.

Embodiment 52: The method of any one of claims 1 to 51, wherein the micro-object is a biological micro-object.

Embodiment 53: The method of any one of claims 1 to 52, wherein the micro-object is a cell, a protein, a peptide, a nucleic acid, or a combination thereof.

Embodiment 54: A method of sampling a micro-object population in a microfluidic device, wherein the microfluidic device comprises a microfluidic circuit material defining a flow region and a chamber comprising a proximal opening fluidically connecting the chamber to the flow region; the method comprises: introducing a first fluidic medium comprising a first reagent to the flow region of the microfluidic device; and allowing the first reagent to diffuse from the flow region into the chamber; introducing a plurality of micro-objects into the flow region of the microfluidic device; allowing the first reagent to diffuse from the chamber to the flow region to contact the plurality of micro-objects; and observing an interaction between the first assay reagent and the plurality of micro-objects.

Embodiment 55: The method of claim 54, wherein the chamber comprises an unswept region.

Embodiment 56: The method of claim 54, wherein the chamber comprises an isolation region and a connection region fluidically connecting the isolation region to the flow region, wherein the isolation region is an unswept region of the microfluidic device.

Embodiment 57: The method of claim 55 or claim 56, wherein the first assay reagent is present within the unswept region before introducing the plurality of micro-object into the flow region.

Embodiment 58: The method of any one of claims 54 to 57, wherein the first assay reagent is allowed to diffuse from the flow region into the chamber for at least about 0.5, 1, 3, 5, 10, 20, 40, 60, 90, 120, 150, 200, or 300 minutes.

Embodiment 59: The method of any one of claims 54 to 58, wherein allowing the first reagent to diffuse from the flow region into the chamber comprises maintaining a continuous perfusion of the first fluidic medium comprising the first reagent.

Embodiment 60: The method of any one of claims 54 to 59, wherein the first fluidic medium comprises the first assay reagent at an initial concentration, and further wherein the initial concentration is at least about 1, about 2, about 4, about 8, about 12, about 16, about 20, about 40, about 100, about 200, or about 2000 times higher than a working concentration of the first assay reagent permitting an interaction between the first assay reagent and the micro-object.

Embodiment 61: The method of nay one of claims 54 to 60, wherein allowing the first assay reagent to diffuse from the flow region into the chamber comprises allowing the first assay reagent to equilibrate between the flow region and the chamber.

Embodiment 62: The method of any one of claims 54 to 61, wherein the first assay reagent is a mixture of reagents.

Embodiment 63: The method of any one of claims 54 to 62, wherein introducing the micro-object comprises introducing a second fluidic medium comprising the micro-object, and stopping a flow of the second fluidic medium after the micro-object is within the flow region.

Embodiment 64: The method of any one of claims 54 to 63, wherein the micro-object substantially has no contact with the first assay reagent until the first assay reagent diffuses from the chamber to the flow region and to contact the micro-object.

Embodiment 65: The method of any one of claims 54 to 64, wherein introducing the micro-object comprises introducing the micro-object to a position within the flow region that is proximal to the opening of the chamber.

Embodiment 66: The method of any one of claims 54 to 65, wherein allowing the first assay reagent to diffuse from the chamber to the flow region comprises contacting the micro-object with the first assay reagent within the flow region.

Embodiment 67: The method of any one of claims 54 to 66, wherein allowing the first assay reagent to diffuse from the chamber to the flow region to contact the plurality of micro-objects within the flow region comprises allowing the first assay reagent to diffuse for at least about 0.5, 1, 3, 5, 10, 20, 40, 60, 90, 120, 150, 200, or 300 minutes.

Embodiment 68: The method of any one of claims 54 to 67, wherein allowing the first assay reagent to diffuse from the chamber to the flow region to contact the plurality of micro-objects within the flow region comprises allowing the first assay reagent to equilibrate between the flow region and the chamber.

Embodiment 69: The method of any one of claims 54 to 68, wherein observing an interaction between the first assay reagent and the plurality of micro-object comprises detecting a first signal associated with the first assay reagent.

Embodiment 70: The method of any one of claims 54 to 69, wherein observing an interaction between the first assay reagent and the plurality of micro-object comprises observing a morphologic change of the micro-object.

Embodiment 71: The method of any one of claims 54 to 70, wherein observing an interaction between the first assay reagent and the plurality of micro-object comprises determining a first percentage or a number of cells of the plurality that interact with the first assay reagent.

Embodiment 72: The method of any one of claims 54 to 71, further comprising: introducing a second assay reagent to the flow region of the microfluidic device; and allowing the second assay reagent to diffuse from the flow region into the chamber.

Embodiment 73: The method of claim 72, wherein the second assay reagent is introduced together with the first assay reagent in the first fluidic medium.

Embodiment 74: The method of claim 72 or claim 73, further comprising observing an interaction between the second assay reagent and the plurality of micro-object.

Embodiment 75: The method of claim 74, wherein observing an interaction between the second assay reagent and the micro-object comprises detecting a second signal associated with the second assay reagent.

Embodiment 76: The method of claim 74 or claim 75, wherein observing an interaction between the second assay reagent and the micro-object comprises determining a second percentage or a number of cells of the plurality that interact with the second assay reagent; and/or determining a third percentage of cells of the plurality that interact with the first assay reagent and the second assay reagent.

Embodiment 77: The method of any one of claims 54 to 76, further comprising introducing a prepolymer composition into the flow region, wherein the prepolymer composition is configured to form an in situ-generated structure within the microfluidic device.

Embodiment 78: The method of claim 77, wherein introducing a prepolymer composition into the flow region comprises introducing a third fluidic medium comprising the prepolymer composition into the flow region; and allowing the prepolymer composition to diffuse into the chamber.

Embodiment 79: The method of claim 78, wherein the prepolymer composition is soluble in the third fluidic medium.

Embodiment 80: The method of any one of claims 77 to 79, further comprising activating solidification of the prepolymer composition thereby forming the in situ-generated structure within the microfluidic device.

Embodiment 81: The method of claim 80, wherein the in situ-generated structure is formed within the chamber, within the flow region, or both.

Embodiment 82: The method of claim 80 or claim 81, wherein the in situ-generated structure is formed in a selected area proximal to the proximal opening of the chamber.

Embodiment 83: The method of any one of claims 77 to 82, wherein the in situ-generated structure has a porosity that restricts passage of the first assay reagent.

Embodiment 84: The method of claim 83, wherein the porosity blocks passage of the first assay reagent.

Embodiment 85: The method of any one of claims 77 to 84, wherein the in situ-generated structure seals the proximal opening of the chamber.

Embodiment 86: The method of claim 85, further comprising unsealing the proximal opening of the chamber.

Embodiment 87: The method of claim 86, wherein unsealing the proximal opening of the chamber comprises removing or reducing the size of the in situ-generated structure so that the proximal opening of the chamber is no longer sealed by the in situ-generated isolation structure.

Embodiment 88: The method of any one of claims 77 to 28, wherein the prepolymer composition comprises: a first polyethylene glycol polymer molecule and a second polyethylene glycol polymer molecule, each comprising a respective polyethylene glycol moiety and a covalently linked reactive moiety Rₓ; a crosslinker molecule comprising a first reactive moiety R_{xP} disposed at a first end of a linker L moiety and a second reactive moiety R_{xP} disposed at a second end of the linker moiety, wherein each of the first and the second crosslinker moiety R_{xP} is configured to be activatable to react with the respective reactive moiety Rₓ of the first and the second polyethylene polymer molecules, wherein the first and the second polyethylene polymer molecule comprises different polyethylene glycol moieties or each comprises a same polyethylene glycol moiety.

Embodiment 89: The method of claim 88, wherein the first polyethylene glycol moiety comprises a 1-arm, 2-arm, 4-arm or 8-arm polyethylene glycol moiety.

Embodiment 90: The method of claim 88 or claim 89, wherein the first polyethylene glycol moiety has a molecular weight from about 500 Da to about 25K Da.

Embodiment 91: The method of any one of claims 88 to 90, wherein the second polyethylene glycol moiety comprises a 1-arm, 2-arm, 4-arm or 8-arm polyethylene glycol moiety.

Embodiment 92: The method of any one of claims 88 to 91, wherein the second polyethylene glycol moiety has a molecular weight from about 500 Da to about 25K Da.

Embodiment 93: The method of any one of claims 88 to 92, wherein the first polyethylene glycol moiety has a molecular weight of about 10K Da and the second polyethylene glycol moiety has a molecular weight of about 20K Da.

Embodiment 94: The method of any one of claims 88 to 93, wherein the first polyethylene glycol molecule and the second polyethylene molecule are present in the composition in a ratio from about 1:100 to about 100:1.

Embodiment 95: The method of claim 94, wherein the ratio of the first polyethylene glycol molecule and the second polyethylene molecule is about 1:1; about 1:2; about 1:3; about 1:4; about 1:5; about 1:6; about 1:7; about 1:8; about 1:9; about 1:10; about 10:1; about 9:1; about 8:1; about 7:1; about 6:1; about 5:1; about 4:1; about 3:1; or about 2:1.

Embodiment 96: The method of any one of claims 88 to 95, wherein the crosslinker molecule comprises a vic-diol.

Embodiment 97: The method of any one of claims 88 to 95, wherein the crosslinker comprises a peptide sequence configured to be a substrate to a protease.

Embodiment 98: The method of claim 97, wherein the crosslinker further comprises a first thiol moiety at a first end of the crosslinker, and a second thiol moiety at the second end of the crosslinker, each configured to react with a norbornenyl moiety of the first or the second polyethylene glycol polymer molecule.

Embodiment 99: The method of claim 97 or claim 98, wherein the peptide sequence comprises GCRDLPRTGGDRCG.

Embodiment 100: The method of any one of claims 97 to 99, wherein the crosslinker comprises a peptide sequence configured to be a tryptase substrate.

Embodiment 101: The method of any one of claims 88 to 95, wherein the crosslinker has a formula: HS-LB4-SH (Formula V); wherein linker backbone LB4 comprises 3 to 200 non-hydrogen atoms selected from any combination of silicon, carbon, nitrogen, oxygen, sulfur and phosphorus atoms.

Embodiment 102: The method of claim 101, wherein the crosslinker is Sodium 2,3-dimercaptopropanesulfonate monohydrate.

Embodiment 103: The method of any one of claims 88 to 102, further comprising an inhibitor configured to inhibit reaction of the crosslinker with the reactive moiety of the first and/or the second polyethylene glycol polymer molecule.

Embodiment 104: The method of claim 103, wherein the inhibitor is sodium ascorbate, MEHQ, or 4-hydroxy TEMPO.

Embodiment 105: The method of any one of claims 54 to 104, wherein the micro-object is a biological micro-object.

Embodiment 106: The method of any one of claims 54 to 105, wherein the micro-object is a cell, a protein, a peptide, a nucleic acid, or a combination thereof.

What is claimed:

1. A method of contacting a micro-object with a reagent within a microfluidic device,
wherein the microfluidic device comprises a microfluidic circuit material defining a flow region and a chamber comprising a proximal opening fluidically connecting the chamber to the flow region;
the method comprises:
introducing a micro-object into the flow region of the microfluidic device; and
allowing a reagent to diffuse from the chamber to the flow region and to contact the micro-object.

2. The method of claim 1, wherein the chamber comprises an unswept region, an isolation region, and a connection region fluidically connecting the isolation region to the flow region, wherein the isolation region is an unswept region of the microfluidic device.

3. (canceled)

4. The method of claim 1, wherein the reagent is present in the chamber before introducing the micro-object into the flow region, wherein the reagent is present in the chamber by:
introducing a first fluidic medium comprising the reagent to the flow region of the microfluidic device; and
allowing the reagent to diffuse from the flow region into the chamber.

5.-6. (canceled)

7. The method of claim 4, wherein allowing the reagent to diffuse from the flow region into the chamber comprises maintaining a continuous perfusion of the first fluidic medium comprising the reagent.

8. The method of claim 4, wherein the first fluidic medium comprises the reagent at an initial concentration, and further wherein the initial concentration is at least about 1, about 2, about 4, about 8, about 12, about 16, about 20, about 40, about 100, about 200, or about 2000 times higher than a working concentration of the reagent permitting an interaction between the reagent and the micro-object.

9. The method of claim 4, wherein allowing the reagent to diffuse from the flow region into the chamber comprises allowing the reagent to equilibrate between the flow region and the chamber.

10. The method of claim 1, wherein the reagent is a mixture of assay reagents.

11. The method of claim 1, wherein the micro-object substantially has no contact with the reagent until the reagent diffuses from the chamber to the flow region and to contact the micro-object.

12. The method of claim 1, wherein allowing the reagent to diffuse from the chamber to the flow region comprises contacting the micro-object with the reagent within the flow region.

13. The method of claim 1, further comprising introducing a prepolymer composition into the flow region, wherein the prepolymer composition is configured to form an in situ-generated structure within the microfluidic device.

14. The method of claim 13, further comprising activating solidification of the prepolymer composition, thereby forming the in situ-generated structure within the microfluidic device, wherein the in situ-generated structure is formed in an area proximal to the proximal opening of the chamber, and comprises a porosity that restricts passage of the reagent.

15.-16. (canceled)

17. The method of claim 13, wherein the in situ-generated structure seals the proximal opening of the chamber.

18. The method of 13, wherein the prepolymer composition comprises:
a first polyethylene glycol polymer molecule and a second polyethylene glycol polymer molecule, each comprising a respective polyethylene glycol moiety and a covalently linked reactive moiety Rₓ;
a crosslinker molecule comprising a first reactive moiety R_{xP} disposed at a first end of a linker moiety L and a second reactive moiety R_{xP} disposed at a second end of the linker moiety L, wherein each of the first and the second crosslinker moiety R_{xP} is configured to be activatable to react with a respective reactive moiety Rₓ of the first and the second polyethylene polymer molecules.

19.-24. (canceled)

25. A method of sampling a micro-object population in a microfluidic device,
wherein the microfluidic device comprises a microfluidic circuit material defining a flow region and a chamber comprising a proximal opening fluidically connecting the chamber to the flow region;
the method comprises:
introducing a first fluidic medium comprising a first reagent to the flow region of the microfluidic device; and
allowing the first reagent to diffuse from the flow region into the chamber;
introducing a plurality of micro-objects into the flow region of the microfluidic device;
allowing the first reagent to diffuse from the chamber to the flow region to contact the plurality of micro-objects; and
observing an interaction between the first assay reagent and the plurality of micro-objects.

26. The method of claim 25, wherein the chamber comprises an unswept region.

27. (canceled)

28. The method of claim 25, wherein allowing the first reagent to diffuse from the flow region into the chamber comprises maintaining a continuous perfusion of the first fluidic medium comprising the first reagent and allowing the first reagent to equilibrate between the flow region and the chamber.

29. The method of claim 25, wherein the first fluidic medium comprises the first reagent at an initial concentration, and further wherein the initial concentration is at least about 1, about 2, about 4, about 8, about 12, about 16, about 20, about 40, about 100, about 200, or about 2000 times higher than a working concentration of the first reagent, permitting an interaction between the first reagent and the micro-object.

30. The method of claim 25, wherein allowing the first reagent to diffuse from the flow region into the chamber comprises allowing the first reagent to equilibrate between the flow region and the chamber.

31. (canceled)

32. The method of claim 25, wherein the plurality of micro-objects substantially has no contact with the first reagent until the first reagent diffuses from the chamber to the flow region and to contact the micro-object.

33.-38. (canceled)

39. A microfluidic device comprising:
a microfluidic circuit material defining a flow region;
a chamber comprising a proximal opening fluidically connecting the chamber to the flow region;
wherein a micro-object is introduced into the flow region of the microfluidic device allowing a reagent to diffuse from the chamber to the flow region and to contact the micro-object.
